(19) United States
(12) Patent Application Publication
(10) Pub. No.: US 2023/0398068 A1
(43) Pub. Date: Dec. 14, 2023

(54) PHARMACEUTICAL COMPOSITION COMPRISING CHLOROQUINE AND USES THEREOF

(71) Applicant: Philip Morris Products S.A., Neuchâtel (CH)

(72) Inventors: Florian Alexis CALVINO, Corcelles (CH); Philippe Alexandre GUY, Nuvilly (CH); Julia HOENG, Corcelles (CH); Aditya Reddy KOLLI, Neuchâtel (CH); Arkadiusz KUCZAJ, Courtaman (CH); Shoaib MAJEED, Neuchâtel (CH); Anatoly MAZUROV, Greensboro, NC (US); Manuel PEITSCH, Peseux (CH); Tanja ZIVKOVIC SEMREN, Bourdry (CH); Marco VAN DER TOORN, Boudry (CH)

(73) Assignee: Philip Morris Products S.A., Neuchâtel (CH)

(21) Appl. No.: 18/044,233

(22) PCT Filed: Sep. 13, 2021
(86) PCT No.: PCT/EP2021/075134

§ 371 Date: Mar. 7, 2023

(30) Foreign Application Priority Data
Sep. 14, 2020 (EP) 20196087.9

(51) Int. Cl. A61K 9/00; A61K 31/4706; A61K 47/10

(52) U.S. Cl. CPC A61K 9/0078; A61K 31/4706; A61K 47/10

(57) ABSTRACT

Pharmaceutical compositions may include chloroquine. More specifically, a pharmaceutical composition comprising chloroquine or pharmaceutically acceptable salt(s) thereof for use in the treatment or prevention of a viral lung infection, preferably caused by Betacoronavirus, including but not limited to 2019-nCoV (coronavirus), SARS-CoV and Middle East respiratory syndrome CoV (MERS-CoV), wherein the pharmaceutical composition is administered by inhalation. An improved delivery into the lungs of a subject with minimum systemic exposure may be achieved.

The invention relates to pharmaceutical compositions comprising chloroquine and uses thereof. More specifically, the invention relates to a pharmaceutical composition comprising chloroquine or pharmaceutically acceptable salts thereof for use in the treatment or prevention of a viral lung infection, preferably caused by Betacoronavirus, including but not limited to 2019-nCoV (coronavirus), SARS-CoV and Middle East respiratory syndrome CoV (MERS-CoV), wherein the pharmaceutical composition is administered by inhalation.

### BACKGROUND

Recent publications including P. Colson et. al., Chloroquine for the 2019 novel coronavirus SARS-CoV-2 Int. J. Antimicrob. Agents (2020); J. Gao, et. al., Breakthrough: chloroquine phosphate has shown apparent efficacy in treatment of COVID-19 associated pneumonia in clinical studies Biosci. Trends (2020); and Liu, J. et. al., Hydroxychloroquine, a less toxic derivative of chloroquine, is effective in inhibiting SARS-CoV-2 infection in vitro. Cell Discov 6, 16 (2020) have brought attention to the possible benefit of chloroquine (CQ), a broadly used antimalarial drug, in the treatment of patients infected by the novel emerged coronavirus (SARS-CoV-2). M. Wang et al., Remdesivir and chloroquine effectively inhibit the recently emerged novel coronavirus (2019-nCoV) in vitro. Cell Res. 2020, 30(3):269-271 provided in vitro studies in which chloroquine was found to block COVID-19 infection at low-micromolar concentration, with a half-maximal effective concentration (EC₅₀) of 1.13 μM. In Yao, X. et. al., In vitro antiviral activity and projection of optimized dosing design of hydroxychloroquine for the treatment of severe acute respiratory syndrome coronavirus 2 (SARS-CoV-2). Clin. Infect. Dis. 2020, the antiviral activity of chloroquine for therapeutic and prophylactic use was tested on the Vero cells infected with a SARS-CoV-2 clinically isolated strain. The EC₅₀ values for chloroquine were 23.90 and 5.47 μM at 24 and 48 hours, respectively.

Putative mechanism of actions against COVID-19 can be summarized as follows. Chloroquine can interfere with the glycosylation of angiotensin-converting enzyme 2 (ACE2) and reduce the binding efficiency between ACE2 on the host cells and the spike protein on the surface of the coronavirus. They can also increase the pH of endosomes and lysosomes, through which the fusion process of the virus with host cells and subsequent replication is prevented. When chloroquine enters antigen-presenting cells, it prevents antigen processing and major histocompatibility complex class II-mediated autoantigen presentation to T cells. The subsequent activation of T cells and expression of CD154 and other cytokines are repressed. In addition, chloroquine disrupts the interaction of DNA/RNA with Toll-like receptors and the nucleic acid sensor cyclic GMP-AMP synthase and therefore the transcription of pro-inflammatory genes cannot be stimulated. As a result, administration of chloroquine not only blocks the invasion and replication of coronavirus, but also attenuates the possibility of cytokine storm as shown in Noël Fa et. al., Pharmacological aspects and clues for the rational use of Chloroquine/Hydroxychloroquine facing the therapeutic challenges of COVID-19 pandemic; Lat Am J Clin Sci Med Technol. 2020 April; 2: 28-34 and Zhou D, et. al., COVID-19: a recommendation to examine the effect of hydroxychloroquine in preventing infection and progression; J Antimicrob Chemother. 2020. As disclosed in Xue J, et. al., Chloroquine Is a Zinc Ionophore. PLoS ONE 9(10), 2014, chloroquine is also a zinc ionophore in A2780 cells, targeting zinc to the lysosomes and from Baric R S, et al., Zn(2+) inhibits coronavirus and arterivirus RNA polymerase activity in vitro and zinc ionophores block the replication of these viruses in cell culture. PLoS Pathog. 2010; 6(11) it was known that zinc has anti-viral properties and can inhibit the replication of coronaviruses in cells.

Chloroquine is a diprotic base with a long terminal elimination half-life in humans (Gustafsson L L, et al., Disposition of chloroquine in man after single intravenous and oral doses. Br J Clin Pharmacol 1983; 15(4): 471-9). By using a physiologically-based pharmacokinetic model for chloroquine phosphate, an oral daily dose of 250 mg until clinical convalescence of COVID-19 has already been the subject of clinical trials (R. Stahlmann, et al., Medication for COVID-19—an overview of approaches currently under study, Arztebl. 117 (13) (2020) 213-219). However, the margin between the therapeutic and toxic dose is narrow and chloroquine poisoning has led to life-threatening cardiovascular disorders as documented in M. Frisk-Holmberg, et al., Chloroquine intoxication [letter] Br. J. Clin. Pharmacol., 15 (1983), pp. 502-503. Use of chloroquine has also led to rare but potentially fatal events, including serious cutaneous adverse reactions (Murphy M, et al., Fatal toxic epidermal necrolysis associated with hydroxychloroquine, Clin Exp Dermatol 2001; 26:457-8); fulminant hepatic failure (Makin A J, et al., Fulminant hepatic failure secondary to hydroxychloroquine, Gut 1994; 35:569-70); and ventricular arrhythmias (especially when prescribed with azithromycin) (Chorin E, Dai M, Shulman E, et al. The QT interval in patients with SARS-Co V-2 infection treated with hydroxychloroquine/azithromycin, medRxiv 2020.04.02). Hence, if high oral doses of chloroquine are necessary to reach higher total unbound lung concentrations, severe side effects and toxicity could arise. Preliminary analysis in Fan et al. (Connecting hydroxychloroquine in vitro antiviral activity to in vivo concentration for prediction of antiviral effect: a critical step in treating COVID-19 patients, Clin. Infect. Diseases, May 2020) extrapolating in vitro to in vivo therapeutic concentrations for treatment of COVID-19, have suggested that the lung interstitial fluid concentrations are well below the in vitro EC₅₀/EC₉₀ values of the literature, making the antiviral effect against SARS-CoV-2 not likely achievable with a safe oral dosing regimen. Further, although virus uptake into the cells lining the respiratory tract has been shown to occur from the apical surface of the respiratory tract that is lined with epithelial lining fluid (Sungnak et al. SARS-CoV-2 entry factors are highly expressed in nasal epithelial cells together with innate immune genes; Nature, 26, 681-687 (2020) the concentrations of orally administered chloroquine in the epithelial lining fluid and epithelial cells of the respiratory tract is not known.

There are various routes by which chloroquine can be administered. Oral treatment with chloroquine has been associated with severe side effects and toxicity. To reach therapeutic concentrations at the target site, i.e. the surface of the lungs, it has been found that relatively high doses of the drug must be administered resulting in a substantial portion of the administered dose accumulating in other organs. Due to increased tissue exposure, the margin between the therapeutic and toxic dose is narrow. Klimke et al. (Hydroxychloroquine as an aerosol might markedly reduce and even prevent severe clinical symptoms after SARS-CoV-2 infection, Medical Hypotheses 142 (2020)), hypothesises that hydroxychloroquine and chloroquine as an aerosol may be an effective way of minimizing systemic concentrations of chloroquine that can lead to severe adverse reactions. However, this paper is purely hypothetical and does not address how, or if, chloroquine can be formulated and administered as an aerosol, nor does it assess whether aerosolised formulations would deliver effective concentrations of chloroquine to the lung whilst minimizing systemic concentrations of the drug.

Thus, there is a clear need for improved pharmaceutical compositions capable of delivering chloroquine, or pharmaceutically acceptable salts thereof, safely and effectively to a subject.

### SUMMARY OF INVENTION

The present invention provides a pharmaceutical composition comprising chloroquine or a pharmaceutically acceptable salt thereof. The pharmaceutical composition comprises a solvent for dissolving the chloroquine or a pharmaceutically acceptable salt thereof. The pharmaceutically acceptable salt of chloroquine may be a phosphate, sulphate, and/or a hydrochloride salt. For example, the pharmaceutically acceptable salt may be chloroquine diphosphate salt (C18H26ClN3·2H3PO4). Preferably, chloroquine is in the form of a free base. The pharmaceutical composition is preferably a liquid comprising 1 mg/mL to 50 mg/mL chloroquine or a pharmaceutically acceptable salt thereof.

Advantageously, the pharmaceutical composition comprising chloroquine or a pharmaceutically acceptable salt thereof may provide an effective formulation for delivery into the lungs of a subject with minimum systemic exposure. More specifically, the pharmaceutical composition may achieve a lung unbound trough concentration of chloroquine in the lung equal to or above EC₅₀ without significantly increasing chloroquine concentrations in other organs, e.g. blood, liver, heart and kidney. This advantageously enables chloroquine or a pharmaceutically acceptable salt thereof to reach a therapeutic pulmonary concentration while maintaining minimum systemic exposure. Further, the pharmaceutical compositions of the invention may advantageously enable delivery of higher doses or prolonged usage of chloroquine or a pharmaceutically acceptable salt thereof to increase or maintain therapeutic pulmonary concentrations.

The pharmaceutical composition of the invention may comprise at least about 1 mg/mL, at least about 5 mg/mL, at least about 10 mg/mL, at least about 15 mg/mL, at least about 20 mg/mL, at least about 25 mg/mL, at least about 30 mg/mL, at least about 35 mg/mL, at least about mg/mL, at least about 45 mg/mL, at least about 50 mg/mL, at least about 55 mg/mL, at least about 60 mg/mL, at least about 65 mg/mL of chloroquine or a pharmaceutically acceptable salt thereof.

The pharmaceutical composition of the invention may comprise no more than about 200 mg/mL, no more than about 190 mg/mL, no more than about 180 mg/mL, no more than about 170 mg/mL, no more than about 160 mg/mL, no more than about 150 mg/mL, no more than about 140 mg/mL, no more than about 130 mg/mL, no more than about 120 mg/mL, no more than about 110 mg/mL, no more than about 100 mg/mL, no more than about 90 mg/mL, no more than about mg/mL, no more than about 70 mg/mL of chloroquine or a pharmaceutically acceptable salt thereof.

The pharmaceutical composition of the invention may comprise about 1 mg/mL to about 200 mg/mL, about 5 mg/mL to about 190 mg/mL, about 10 mg/mL to about 180 mg/mL, about 15 mg/mL to about 170 mg/mL, about 20 mg/mL to about 160 mg/mL, about 25 mg/mL to about 150 mg/mL, about 30 mg/mL to about 140 mg/mL, about 35 mg/mL to about 130 mg/mL, about 40 mg/mL to about 120 mg/mL, about 45 mg/mL to about 110 mg/mL, about 50 mg/mL to about 100 mg/mL, about 55 mg/mL to about 90 mg/mL, about 60 mg/mL to about 80 mg/mL, about 65 mg/mL to about 70 mg/mL of chloroquine or a pharmaceutically acceptable salt thereof.

Alternatively, the pharmaceutical composition of the invention may comprise 1 mg/mL to 50 mg/mL, 10 mg/mL to 45 mg/mL, 20 mg/mL to 40 mg/mL, 30 mg/mL to 35 mg/mL of chloroquine or a pharmaceutically acceptable salt thereof. The pharmaceutical composition may comprise any range from the given endpoints, for example, but not limited to, 10 mg/mL to 40 mg/mL, 20 mg/mL to 30 mg/mL and/or 30 mg/mL to 50 mg/mL. Advantageously, this concentration range may provide a therapeutically effective dose for delivery into the lungs which requires less chloroquine compared with known compositions.

Preferably the pharmaceutical composition may comprise chloroquine or a pharmaceutically acceptable salt thereof, and a solvent, wherein the pharmaceutical composition comprises 1 mg/mL to 50 mg/mL chloroquine or a pharmaceutically acceptable salt thereof.

Preferably, the pharmaceutical composition may comprise a solvent selected from propylene glycol, glycerine, and water or combinations thereof. Propylene glycol and its IUPAC name propane-1,2-diol may be used interchangeably. The solvent may also be propane-1,3-diol. Other pharmaceutically acceptable solvents may be used provided they dissolve chloroquine at 40° C. and atmospheric pressure (˜100 kPa) and are stable at temperatures of about 150 to about 300° C.

The solvent may comprise 20%, 25%, 30% 45%, 50%, 55%, 60%, 65%, 70%, 75%, 80%, 85%, 90%, 95% or 100% of propylene glycol; 0%, 5%, 10%, 15%, 20%, 25%, 30%, 35%, 40%, 45%, 50%, 55%, 60% of glycerine; and/or 0%, 5%, 10%, 15%, 20%, 25%, 30%, 35%, 40% of water, or any combination thereof.

Where the solvent comprises a combination of propylene glycol and water, the solvent may comprise about 85%, about 90%, about 95% of propylene glycol and about 5%, about 10%, about 15% of water. Preferably the solvent may comprise about 15% of water and about 85% of propylene glycol. More preferably, the solvent may comprise about 5% of water and about 95% of propylene glycol. Most preferably the solvent may comprise about 10% of water and about 90% of propylene glycol.

The solvent may comprise about 90% of propylene glycol and about 10% of glycerine.

Where the solvent comprises a combination of propylene glycol, glycerine and water, the solvent may comprise at least about 45% of propylene glycol, at least about 15% of glycerine, and at least about 5% of water.

Where the solvent comprises a combination of propylene glycol, glycerine and water, the solvent may comprise no more than about 75% of propylene glycol, no more than about 45% of glycerine, and no more than about 10% of water.

Where the solvent comprises a combination of propylene glycol, glycerine and water, the solvent may comprise about 45% to about 75% of propylene glycol, about 15% to about 45% of glycerine, and about 5% to about 10% of water. Preferably the solvent may comprise about 10% of water, about 45% of propylene glycol, and about 45% of glycerine. More preferably, the solvent may comprise about 10% of water, about 75% of propylene glycol, and about 15% of glycerine. Most preferably the solvent may comprise about 5% of water, about 75% of propylene glycol, and about 20% of glycerine.

Advantageously by varying the ratio of solvents the solubility and/or stability of chloroquine or a pharmaceutically acceptable salt thereof may be improved.

As used herein, the terms “glycerine” and “glycerol” are synonyms of each other and may therefore be used interchangeably.

In a preferred embodiment, the pharmaceutical compositions of the invention do not comprise a propellant. A propellant may comprise, but is not limited to, one or more of tetrafluoroethane, pentafluoroethane, hexafluoroethane, heptafluoroethane, heptafluoropropane.

Preferably, the pharmaceutical composition may be thermally aerosolized. Surprisingly, chloroquine or a pharmaceutically acceptable salt thereof transfers into a liquid aerosol by thermal vaporization. Thus, the thermally aerosolized pharmaceutical composition may be used to provide an effective dose of chloroquine or a pharmaceutically acceptable salt thereof into the lungs with minimum systemic exposure. The thermal vaporization may be performed at high temperatures, for example between 100° C. and 300° C., preferably 150° C. and 250° C., more preferably between 200° C. and 220° C. Advantageously, thermal vaporization may provide a more suitable particle size for delivery to the lung compared with non-thermal liquid aerosolization (e.g. nebulization) thus providing the additional benefit of improved delivery of chloroquine to the lung without decomposition of chloroquine or a pharmaceutically acceptable salt thereof. Furthermore, thermal vaporization may provide high transfer efficiency from the pharmaceutical composition to the thermally aerosolized pharmaceutical composition. The transfer efficiency may be from 60-100%; 70-100%; 80-100% or 90-100%. Advantageously, a high transfer efficiency may provide a high loaded aerosolized dose for inhalation thus reducing the number of inhalations necessary to deliver and effective dose of chloroquine or a pharmaceutically acceptable salt thereof to the lung. The pharmaceutical composition according to the invention may be for thermal aerosolization. Alternatively, the pharmaceutical composition according to the invention is thermally aerosolized.

The invention also provides a pharmaceutical composition comprising chloroquine or a pharmaceutically acceptable salt thereof for use in the treatment or prevention of a viral lung infection. The treatment may include prophylactic and/or therapeutic treatment. For example, the treatment may include improving the condition of, and/or curing, a subject suffering from a viral lung infection. The treatment may also include prevention of a viral lung infection, for example stopping the progress of a viral lung infection or stopping a viral lung infection from arising. The viral lung infection may be pneumonia or an inflammation caused by a viral infection. The viral lung infection may affect one or both lungs.

The pharmaceutical composition comprising chloroquine or a pharmaceutically acceptable salt thereof may be used in the treatment or prevention of a viral lung infection. The pharmaceutical composition for use in the treatment or prevention of a viral lung infection may be administered by inhalation, preferably oral inhalation. As used herein, the term “inhalation” describes the action of breathing into the lungs of a subject. Although oral inhalation is preferred, inhalation may also include nasal inhalation or inhalation through intubation, for example by inserting an endotracheal tube through the mouth or via tracheostomy. Advantageously, the administration of a pharmaceutical composition according to the invention by inhalation enables chloroquine or a pharmaceutically acceptable salt thereof to be delivered directly to the lungs of a subject thus limiting systemic exposure. In contrast to solid oral administration, directly delivering chloroquine or a pharmaceutically acceptable salt thereof to the lungs may provide the additional advantage of requiring administration of less chloroquine to achieve a comparable therapeutic effect. Additionally, the administration by inhalation may advantageously provide the required total lung unbound concentrations without increasing the undesirable accumulation of chloroquine or pharmaceutically acceptable salts thereof in organs other than the lungs, e.g. heart, liver, kidney. This is because doses of inhaled chloroquine or a pharmaceutically acceptable salt thereof may reach a therapeutic pulmonary concentration of chloroquine with minimum systemic exposure. In turn, the use of a pharmaceutical composition comprising chloroquine or a pharmaceutically acceptable salt thereof in the treatment or prevention of a viral lung infection may not be limited in its ability to deliver higher doses or prolonged usage to further increase lung concentrations. As an additional benefit, the administration by inhalation provides greater flexibility by enabling dosing regimens to be individualized to a subject.

Preferably, the pharmaceutical composition comprising chloroquine or a pharmaceutically acceptable salt thereof may be for use in the treatment of a viral lung infection caused by Betacoronavirus, including but not limited to 2019-nCoV (coronavirus), SARS-CoV and Middle East respiratory syndrome CoV (MERS-CoV). Preferably, the pharmaceutical composition comprising chloroquine or a pharmaceutically acceptable salt thereof may be for use in the treatment or prevention of COVID-19. COVID-19 may be caused by coronavirus, e.g. SARS-CoV-2. More specifically, COVID-19 may be caused by the novel coronavirus (2019-nCoV), which is closely related to severe acute respiratory syndrome CoV (SARS-CoV).

Preferably, the pharmaceutical composition comprising chloroquine or a pharmaceutically acceptable salt thereof for use in the treatment or prevention of a viral lung infection may be administered as a daily dose. As used herein, the term “daily” is understood to mean every day, for example within a 24 hour period. The daily dose relates to the total amount of chloroquine or a pharmaceutically acceptable salt thereof administered to a subject within a 24-hour period.

The “daily dose” may be a “loading dose”; a “maintenance dose” or combinations thereof. As used herein, the term “loading dose” relates to a dose that rapidly reaches an effective pulmonary concentration, e.g. EC₅₀ or EC₉₀, of chloroquine in a subject. The term “maintenance dose” relates to a dose that is capable of maintaining an effective pulmonary concentration, e.g. EC₅₀ or EC₉₀, of chloroquine in a subject over prolonged a period of time, e.g. greater than 3 days. A maintenance period may be any period necessary for the subject to substantially recover from the viral lung infection and may be from 1 week to multiple years. For example, a maintenance period may be selected from 1, 2, 3 or 4 weeks; 1, 2, 3, 4, 5, 6, 7, 8, 9, 10, 11 or 12 months; or 1 or 2 years. In exceptional circumstances, the maintenance period may be great than 2 years.

Preferably, the daily dose may be less than 450 mg, less than 350 mg, less than 250 mg, less than 150 mg or less than 50 mg. More preferably, the daily dose may comprise 0.001 mg to mg, 0.005 mg to 15 mg, 0.01 mg to 10 mg, 0.05 mg to 8 mg, 0.1 to 6 mg, or 0.15 mg to 5 mg of chloroquine or a pharmaceutically acceptable salt thereof. The daily dose may comprise any range from the given endpoints, for example, but not limited, to 0.005 mg to 20 mg, 0.005 mg to 8 mg, 0.1 mg to 20 mg. Advantageously, these daily doses may provide a therapeutic pulmonary concentration of chloroquine or a pharmaceutically acceptable salt thereof with minimum systemic exposure.

Preferably, the loading dose may comprise 1 mg to 20 mg, 2 mg to 15 mg, 3 mg to 10 mg, 4 mg to 8 mg, 4.5 mg to 6 mg of chloroquine or a pharmaceutically acceptable salt thereof. The loading dose may comprise any range from the given endpoints. Advantageously, the loading dose may provide a therapeutic concentration initially required to rapidly achieve an effective pulmonary concentration, e.g. EC₅₀ or EC₉₀, of chloroquine required for the treatment of a viral lung infection.

Preferably, the maintenance dose may comprise 0.001 mg to 10 mg, preferably 0.005 mg to 9 mg, preferably 0.01 mg to 8 mg, preferably 0.05 mg to 7 mg, preferably 0.1 to 6 mg, preferably mg to 5 mg of chloroquine or a pharmaceutically acceptable salt thereof. The maintenance dose may comprise any range from the given endpoints. Advantageously, the maintenance dose may maintain the effective pulmonary concentration, e.g. EC₅₀ or EC₉₀, of chloroquine over a prolonged a period of time required for the treatment or prevention of a viral lung infection.

Preferably, at least one loading doses is followed by at least one maintenance doses. More preferably, the loading dose is higher than the daily maintenance dose.

Preferably, the daily dose may be administered in at least one session. When the daily dose comprises at least two sessions, the sessions may be separated by intervals of twelve hours, ten hours, eight hours, seven hours, or six hours. Advantageously, the administration over at least two sessions provides for a more controlled administration of chloroquine or a pharmaceutically acceptable salt thereof. Preferably, the daily dose comprises three sessions separated by intervals of six hours.

The session may comprise administration of at least one fixed dose. As used herein, the term “fixed dose” defines a specific dose of chloroquine or a pharmaceutically acceptable salt thereof dispensed from an aerosol generating device in a single inhalation. One session may comprise 1-20, 2-19, 3-18, 4-17, 5-16, 6-15, 7-14, 8-13, 9-12, 10-11 fixed doses. The session may comprise any range of fixed doses from the given endpoints.

Preferably, the fixed dose may be a metered dose. The term “metered dose” defines a fixed dose of chloroquine or a pharmaceutically acceptable salt thereof dispensed from an aerosol generating device in a single inhalation, wherein the fixed dose is regulated by the aerosol generating device. Advantageously, this may ensure that a therapeutically effective dose of chloroquine or a pharmaceutically acceptable salt thereof is administered in a controlled and consistent manner.

Preferably, the pharmaceutical composition may be administered as a liquid aerosol. Preferably, the pharmaceutical composition may be thermally aerosolized. The liquid aerosol may be provided by thermally vaporizing the pharmaceutical composition at high temperatures, for example between 100° C. and 300° C., preferably 150° C. and 250° C., more preferably between 200° C. and 220° C. Advantageously, the liquid aerosol may provide a suitable particle size with the additional benefit of providing a high effective drug concentration in the lung without decomposing chloroquine or a pharmaceutically acceptable salt thereof. Further, the liquid aerosol may contain a high concentration of chloroquine or a pharmaceutically acceptable salt thereof as a result of the high transfer efficiency from the pharmaceutical composition. Advantageously, this may provide a high loaded aerosolized dose for inhalation thus reducing the number of inhalations necessary to deliver and effective dose of chloroquine or a pharmaceutically acceptable salt thereof to the lung.

Preferably, the Mass Median Aerodynamic Diameter (MMAD) of the liquid aerosol may be 1 to 10 μm, preferably from 1 to 5 μm, more preferably 1 to 3 μm, for example, 1 μm, 2 μm and/or 3 μm, or any fraction in between. Further, the geometric standard deviation (GSD) may be 1 to 3, preferably 1 to 1.5. Advantageously, this may provide a suitable particle size and/or distribution for the chloroquine or a pharmaceutically acceptable salt to reach and deposit in the alveoli of a subject in order to provide an effective concentration of chloroquine in the lung.

As used herein, the term “deposited dose” defines the amount of chloroquine or a pharmaceutically acceptable salt thereof deposited in the lung. Preferably the deposited dose of the pharmaceutical composition of the present invention may be 20 to 70% of the fixed dose. More preferably the deposited dose may be 25 to 60%, 30 to 50%, 35 to 40% of the fixed dose. Advantageously this may enable more efficient delivery of chloroquine directly to the lung thus reducing the number of inhalations required by a subject and reducing the risk of systemic exposure resulting from unintentional ingestion of the liquid aerosol during inhalation.

Preferably, the pharmaceutical composition comprising chloroquine or a pharmaceutically acceptable salt thereof may be for use in the treatment or prevention of a viral lung infection in a mammalian or avian subject. The mammalian subject may be a human subject, a primate, a rodent, a bat, a carnivore, for example a dog or a feline, for example a domestic cat.

According to the invention, the subject may be at risk of having COVID-19. For example, the subject may also be at risk of having COVID-19 due to traveling, in particular traveling from high-risk areas, direct contact with a subject positively tested for COVID-19 and/or visiting public or shared spaces. The subject may be at risk due to a physical co-morbidity, such as cancer, chronic kidney disease COPD (chronic obstructive pulmonary disease), immunocompromised state (weakened immune system) from solid organ transplant, obesity (body mass index [BMI] of 30 or higher), serious heart conditions, such as heart failure, coronary artery disease, or cardiomyopathies, sickle cell disease, and/or type 2 diabetes mellitus. The subject may also be at risk due to asthma (moderate-to-severe), cerebrovascular disease (affects blood vessels and blood supply to the brain), cystic fibrosis, hypertension or high blood pressure, immunocompromised state (weakened immune system) from blood or bone marrow transplant, immune deficiencies, HIV, use of corticosteroids, or use of other immune weakening medicines, neurologic conditions, such as dementia, liver disease, pregnancy, pulmonary fibrosis (having damaged or scarred lung tissues), smoking, thalassemia (a type of blood disorder), and/or type 1 diabetes mellitus. Further, the subject may also be at risk due to being above 50 years of age, above 60 years of age, above 70 years of age, above 80 years of age, and/or above 90 years of age.

The subject may show symptoms of having COVID-19, for example, fever, dry cough, loss of taste and/or tiredness; and/or less common symptoms, for example, aches and pains, sore throat, diarrhoea, conjunctivitis, headache, loss of taste or smell, and/or a rash on the skin, or discolouration of fingers or toes.

The subject may be COVID-19 positive as confirmed by, for example, PCR testing.

Advantageously, the pharmaceutical composition comprising chloroquine or a pharmaceutically acceptable salt thereof administered by inhalation for use in the treatment or prevention of a viral lung infection in a subject may particularly benefit a subject at risk of having COVID-19 and/or showing symptoms of having COVID-19 and/or having tested positive for COVID-19 as it is possible to titre doses such that they are suitable for treatment or prevention without unnecessarily exposing the subject to potentially toxic systemic levels of chloroquine.

Preferably, the pharmaceutical composition comprising chloroquine or a pharmaceutically acceptable salt thereof may be for use in the treatment or prevention of a viral lung infection, wherein the pharmaceutical composition is administered by inhalation and wherein the total lung unbound concentrations of chloroquine or a pharmaceutically acceptable salt thereof in the subject is between 100 ng/mL to 3000 ng/mL.

In vitro studies have shown that an increase in chloroquine duration of exposure from 24 h to 48 h has lowered the EC₅₀ value (Yao et al. 2020). This indicates that duration of exposure or time above EC₅₀ or EC₉₀ could be the pharmacokinetic driver for chloroquine in humans. So, it is necessary to get the human unbound total lung concentrations to concentrations above EC₉₀ rapidly and maintain these concentrations for a longer duration to obtain efficacy.

Preferably, the pharmaceutical composition comprising chloroquine or a pharmaceutically acceptable salt thereof may be for use in the treatment or prevention of a viral lung infection, wherein the plasma concentration of chloroquine or a pharmaceutically acceptable salt thereof in the subject is below 800 ng/mL, preferably below 600 ng/mL, more preferably below 500 ng/mL, most preferably below 400 ng/mL. Limiting the plasma concentration of chloroquine or a pharmaceutically acceptable salt thereof may ensure an effective dosage regimen which is below warning levels of systemic toxicity (800 ng/mL) and even below levels at which no adverse reactions have been observed (400 ng/mL).

The present invention also provides an aerosol generating device comprising the pharmaceutical composition of the invention. Preferably, the aerosol generating device comprises: a cartridge comprising the pharmaceutical composition; a heating element for heating the pharmaceutical composition; a power supply for supplying power to the heating element; and a mouthpiece. The aerosol generating device may be an oral delivery device adapted for delivery of a liquid aerosol to a subject. The aerosol generating device may comprise a heating element; a power supply; and a mouthpiece. The aerosol generating device may comprise a cartridge comprising the pharmaceutical composition of the invention. Advantageously, the aerosol generating device may provide a convenient delivery means of the pharmaceutical composition of the invention.

The aerosol generating device may be a stand-alone device or it may form part of another device such as a ventilator.

Preferably, the aerosol generating device may comprise an element for metering a fixed dose of the pharmaceutical composition. Advantageously, the element for metering a fixed dose may ensure that a therapeutically effective dose of chloroquine or a pharmaceutically acceptable salt thereof is administered in a controlled and consistent manner. Additionally, the metering element may provide greater flexibility and reliability allowing for dosing regimens to be individualized to a subject.

The invention also provides a cartridge for use in an aerosol generating device, the cartridge comprising the pharmaceutical composition according to the invention. Preferably, the cartridge may comprise an atomiser configured to generate an aerosol from the pharmaceutical composition. Preferably, the cartridge may be replaceable.

The invention also provides a method for forming an aerosol, the aerosol comprising the pharmaceutical composition according to the invention, wherein the method comprises a step of vaporizing the pharmaceutical composition to form an aerosol. Preferably, the step of thermally vaporizing the pharmaceutical composition according to the invention occurs between 100° C. and 300° C., preferably 150° C. and 250° C., more preferably between 200° C. and 220° C.

The invention also provides a method of treating a viral lung infection, preferably caused by Betacoronavirus, for example, 2019-nCoV (coronavirus), SARS-CoV and Middle East respiratory syndrome CoV (MERS-CoV), comprising administrating by inhalation a pharmaceutical composition according to the invention.

The method also provides a use of chloroquine or a pharmaceutically acceptable salt thereof for the manufacture of a medicament for the treatment of a viral lung infection, preferably caused by Betacoronavirus, for example, 2019-nCoV (coronavirus), SARS-CoV and Middle East respiratory syndrome CoV (MERS-CoV), wherein the medicament is administered by inhalation, preferably oral inhalation.

The invention further provides an aerosol-generating system comprising: the pharmaceutical composition according to the invention; and an atomiser configured to generate an aerosol from the pharmaceutical composition.

### LIST OF FIGURES

FIG. 1 shows the stages of exposure, onset of illness and scope for pulmonary delivery of chloroquine for COVID-19 treatment.

FIG. 2 provides (A) a procedure used to test aerosolization of chloroquine (CQ) using a mesh system wherein CQ was solubilized in propylene glycol (PG) at a final concentration of 40 mg/mL; (B) the instrumental set-up to measure the aerosolization of CQ using the aerosol generator device described in WO2018153608 (A1) coupled to a PDSP pump connected to SUPER SESI interfaced with Q Exactive HF high resolution accurate mass spectrometer; and (C) an approach for transfer rate assessment.

FIG. 3 shows a schematic layout of an aerosol generating device connected to Aerodynamic Particle Sizer for measuring aerosol particle diameters.

FIG. 4 shows the generation and characterization of chloroquine (CQ) and hydroxychloroquine (HCQ) aerosol from thermal aerosolization including (A) the particle size measurements; and (B) the amount of CQ and HCQ transferred per puff from the device.

FIG. 5 provides LC-HR-MS analyses of a blank sample and aerosol sample of chloroquine (CQ) trapped in Cambridge pad filter. (A) blank sample showing few peaks originating from the column (background contamination), (B) total ion current of CQ sample showing a clear peak at retention time of 2.55 min, (C) accurate mass extraction of chloroquine (m/z 320.18880) from CQ sample (5 ppm mass tolerance).

FIG. 6 provides a schematic representing in vitro aerosol generation and the exposure system. The generated aerosol is passed through (A) the dilution chamber without any dilution into (B) the exposure chamber having (C) trumpet-like outlets to the cell culture inserts which contain 3D organotypic human bronchial airway cultures at air-liquid interface on a porous membrane and culture medium at the bottom.

FIG. 7 shows the in vitro assessment of functional activity and cell viability: (A) Ciliary Beating Frequency (CBF), (B) Cilia beating active area, (C) Cellular ATP levels and (D) Transepithelial electrical resistance (TEER) of 3D in cell cultures before (light grey bars) and after (dark grey bars) exposure to different concentrations of chloroquine. Data are presented as mean (bars) of 3 technical replicates (dots)±95% confidence interval.

FIG. 8 shows simulated transport kinetics of (A) hydroxychloroquine in human bronchial epithelial culture (HBEC) at ALI exposed to 25, 50 and 100 puffs of hydroxychloroquine aerosol (simulation-lines, experimental mean—dots and error bars represent the 95% confidence interval) and (B) chloroquine in isolated perfused mouse lung with P-gp efflux transporter (triangles—experimental data, dashed line—simulated data) and P-gp efflux transporter knockout lung (dots—experimental data, solid line—simulated data). Chloroquine experimental data was obtained from Price et al. (The Differential Absorption of a Series of P-Glycoprotein Substrates in Isolated Perfused Lungs from Mdr1a/1b Genetic Knockout Mice can be Attributed to Distinct Physico-Chemical Properties: an Insight into Predicting Transporter-Mediated, Pulmonary Specific Disposition. Pharm Res. 2017; 34(12):2498-2516)

FIG. 9 provides a schematic of (A) the inhalation PBPK model for chloroquine and hydroxychloroquine with (B) detailed airway tract compartments. GI is the gastrointestinal tract.

FIG. 10 shows pharmacokinetic profiles of chloroquine (CQ) in rats upon 10 mg/kg i.p administration where GI represents the gastrointestinal tissue and the dots are experimental data obtained from Adelusi et al., Kinetics of the distribution and elimination of chloroquine in the rat, Gen Pharmacol, 1982; 13(5):433-7

FIG. 11 shows simulated human pharmacokinetic profiles of (A) 300 mg i.v chloroquine (CQ), (B) 600 mg oral CQ where the dots represent experimental data obtained from Gustafsson et al.

FIG. 12 shows a model of predicted inhalation and oral dosing regimens for chloroquine (CQ) where the horizontal dashed lines represent the in vitro EC₅₀ (362 ng/mL) and EC₉₀ (2208 ng/mL) values from Wang et al.

FIG. 13 shows simulated chloroquine (CQ) concentrations in different compartments representing a human lung. ‘APAmucus’ represents surfactant concentrations in the mucus. ‘Lung_Interstitial_Free’ represents the unbound concentrations in lung interstitial space as shown in FIG. 9B, ‘Lung_Cellular_Free’ represents the unbound intracellular cytosolic concentrations in lung, ‘Lung_Free’ represents the unbound total lung concentrations.

FIG. 14 shows simulated chloroquine (CQ) concentrations in different tissues for multiple dosing regimens. The horizontal dashed lines represent the 400 ng/mL plasma concentration cut off threshold showing no adverse events and 800 ng/mL is the warning limit where 80% subjects 5 showed adverse events based on values from Frisk et al. (Chloroquine serum concentration and side effects: evidence for dose-dependent kinetics. Clin Pharmacol Ther. 1979; 25(3):345-350.) and Cui et al. (Dose selection of chloroquine phosphate for treatment of COVID-19 based on a physiologically based pharmacokinetic model. Acta Pharmaceutica Sinica B. 2020)

FIG. 15 shows model predicted chloroquine concentrations in blood, total unbound concentrations in lung (Lung_Free) and unbound pulmonary alveolar region concentrations (PA_Free) for monodisperse and polydisperse aerosols with different aerosol particle sizes. MMAD, mass median aerodynamic diameter and GSD, geometric standard deviation.

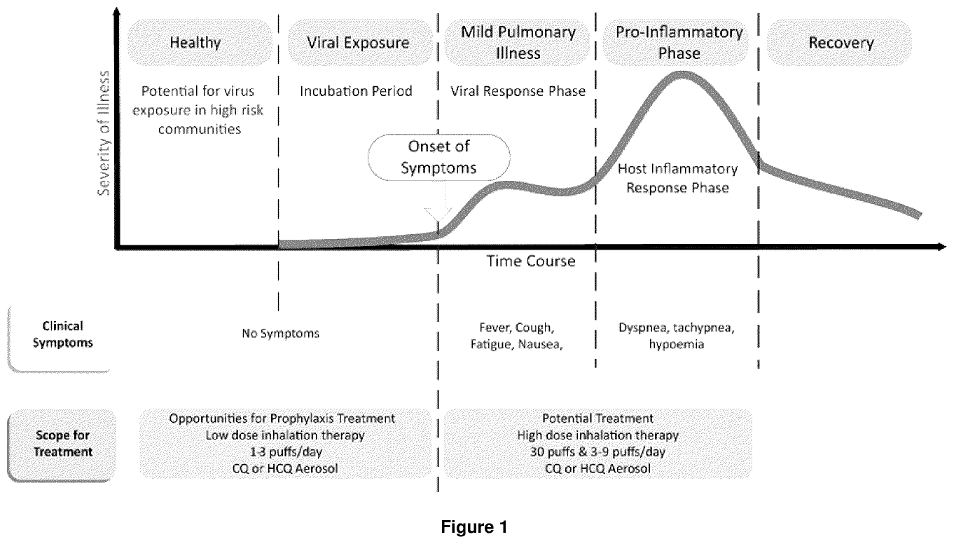

### DETAILED DESCRIPTION

By delivering a low-dose of chloroquine through a non-systemic route, adverse drug reactions can be markedly reduced compared to an oral route of administration while reaching effective therapeutic concentrations in the lung. This increase in tolerability enables a broader use for prevention and treatment, particularly after contact with an infected person, which can be advantageous especially for high-risk, often multi-morbid and elderly patients. Further, the pulmonary route of delivery will facilitate higher lung lining fluid/epithelial lining fluid (ELF) concentrations of chloroquine to reach therapeutic levels. This is beneficial compared to oral administration as the postulated mechanism of viral uptake from the respiratory tract may be interfered with by altering the pH of the airway surface liquid (ASL), thereby inhibiting the endosomal uptake mechanism and intracellular lysosomal release for viral replication. The increased pH in ELF also interferes with the glycosylation of angiotensin-converting enzyme 2 (ACE2) to reduce the binding efficiency between ACE2 on the host cells and the spike protein on the surface of the coronavirus.

Various methods are known for generating aerosol from a liquid pharmaceutical formulation including the use of hydrofluoroalkane propellants (e.g. HFAs 134a and 227); nebulisers; and technologies utilising a mechanically induced pressure gradient such as the Respimat® inhaler. The aerosols described herein are generated using a thermal aerosolization processes in which the liquid pharmaceutical composition is heated for effective (large drug concentration without decomposition products) evaporation and subsequently cooled in order to nucleate and condense aerosol particles from the supersaturated vapours. This approach, under controlled thermal conditions, enables the generation of micrometer and even sub-micrometer aerosol size particles that are easily inhalable and able to penetrate deep into the lung. Using any suitable aerosol generator, liquid aerosol particle sizes with a median mass aerodynamic diameter (MMAD) of between 1-5 μm may be produced. For example, in one embodiment an aerosol generator as described in WO2018153608A1 may be used. Using the thermal aerosol generator described therein, an aerosol with MMAD of 1.3 μm can be generated, having a geometric standard deviation of 1.5. Furthermore, it has been found that the thermal aerosolization process of the present invention has a transfer efficiency of chloroquine from liquid composition to liquid aerosol of about 80%, using a liquid pharmaceutical composition containing mg/mL CQ when delivering 0.15 mg metered dose.

The following describes the tests and measurements performed to assess and characterise the invention.

### Compound Synthesis and Aerosol Formulation

Chloroquine and hydroxychloroquine were synthesized according to published procedures at WuXi AppTec (Wuhan, China). The synthesized chloroquine (CQ) and hydroxychloroquine (HCQ) had a purity of 98.3% and 99.7%. The solubility of CQ in propylene glycol (PG) was evaluated by preparing formulations at various concentrations and assessing their solubility by performing liquid chromatography-high resolution-mass spectrometry (LC-HR-MS). The solubility of CQ in PG was measured at 40° C. and atmospheric pressure (˜100 kPa)—Table 1.

TABLE 1
Solubility of chloroquine in propylene glycol.

| | Calculated concentration¹ | Accuracy² |
|---|---|---|
| Drug | (mg/mL) | (%) |
| Chloroquine | 1.0 | 96 |
| | 5.4 | 84 |
| | 10.1 | 94 |
| | 20.2 | 80 |
| | 40.7 | 81 |

¹Calculated concentration based on the mass of chloroquine and volume of propylene glycol
²Accuracy is the ratio of measured concentration to calculated concentration

### Aerosol Generation and Characterization

A liquid formulation containing a solution of chloroquine in propylene glycol at a concentration of 40 mg/mL was prepared and filled into a consumable cartridge.

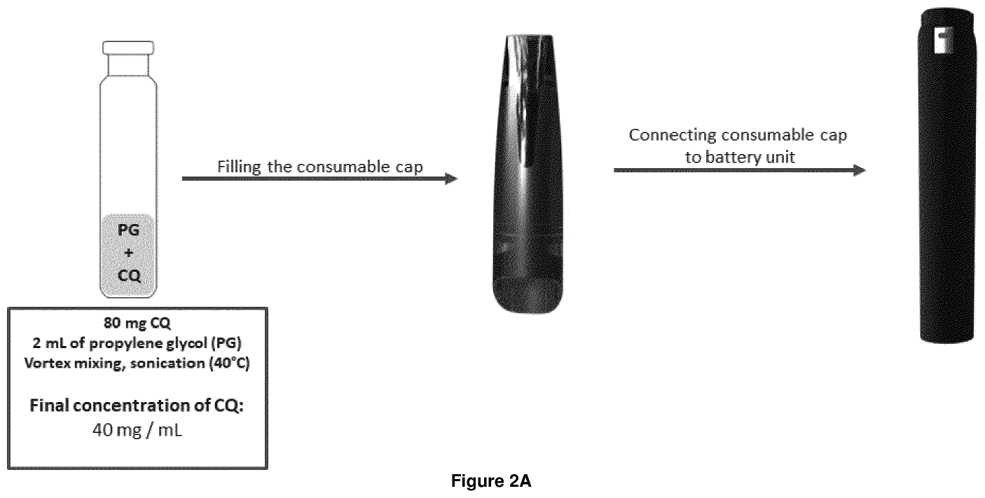

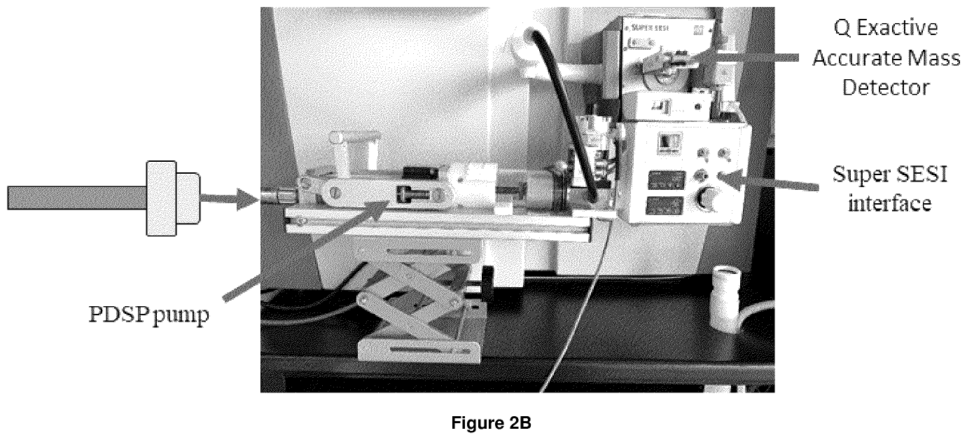

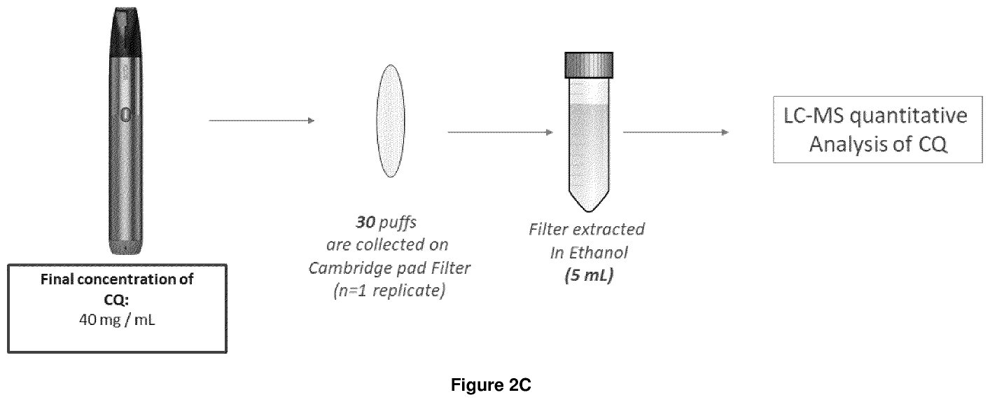

The liquid formulation was nebulized using a device comprising a mesh heating element as described in WO2018153608 (A1). Aerosol from the liquid formulation was generated by thermal aerosolization. The temperature of the heater was maintained between 200-220° C. A programmable dual syringe pump (PDSP) with a simulated inhalation regimen of 55 mL delivered in 3 sec puff duration and 30 sec interval (including the subsequent discharge from the pump during piston down stroke) was used to transfer the aerosol. The PDSP pump was attached to a SUPER SESI (Fossilion Technologies, Madrid, SP) interfaced with Q Exactive HF system (Thermo Fisher Scientific, Waltham, MA, USA) as shown in FIG. 2.

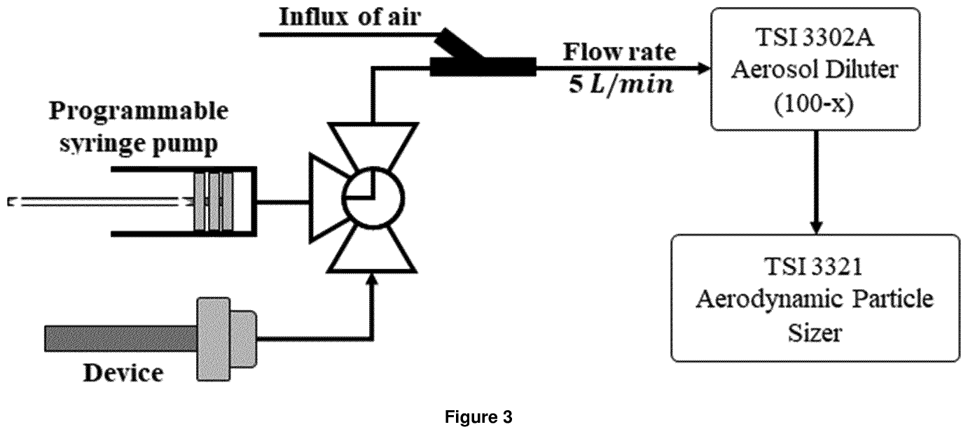

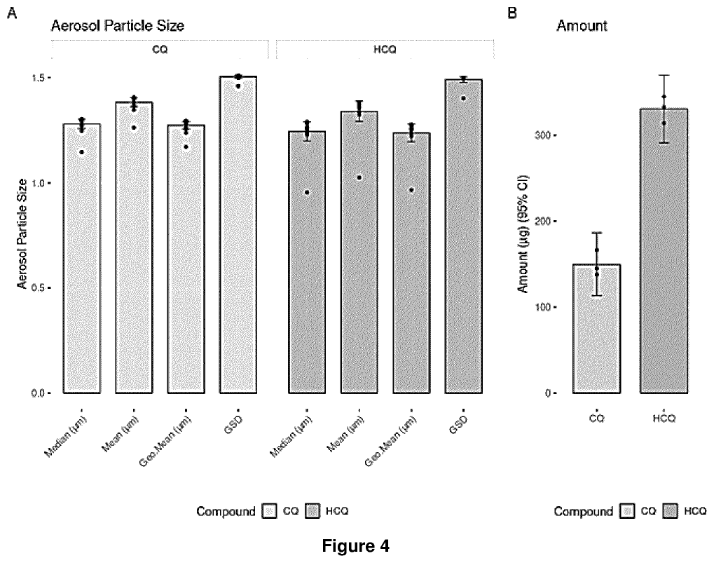

The particle size distribution of the aerosols was measured using the TSI 3321 Aerodynamic Particle Sizer (APS, TSI Incorporated, Shoreview, MN, USA). To reach the operational flowrate of 5 L/min and to stay within the limits of detection concerning large particle number densities obtained in the experiment, the single programmable syringe pump was connected with a 3302A Aerosol Diluter (TSI Incorporated), upstream of the APS using a 30 cm conductive tube with a 1-cm inner diameter (FIG. 3). To avoid the build-up of negative pressure in the connection a Y-piece, open towards the surroundings, was installed between the syringe pump and the APS. In this configuration, the difference between the volume flow supplied by the syringe pump and the volume flow required by the APS is compensated by the influx of surrounding air into the system. The samples were diluted a 100-fold using a 3302A Aerosol Diluter (TSI Incorporated) upstream of the APS to maintain appropriate flows for the particle size measurements and chemical characterization. The discharging periods from the syringe pump were varied between 3 s (average 1.1 L/min) for the APS and 8 s (average 0.41 L/min) for the in vitro aerosol delivery. The aerosol particle sizes had a median aerodynamic diameter of 1.3 μm and a geometric standard deviation (GSD) of 1.5 (FIG. 4A).

### Assessment of Aerosol Characteristics

### Transfer Efficacy/Aerosol Solubility

Using the described aerosol generation and characterization setup, the aerosol produced from the device was pushed through the Cambridge filter pad connected to an impinger filled with 5 mL of ethanol to assess the transfer amount of chloroquine from the liquid to the aerosol. Compound extraction from the Cambridge filter pads was performed by adding 5 mL of ethanol from the impinger and another 5 mL of fresh ethanol to the filter pad. The two fractions were combined (total volume of 10 mL) for quantification. Chemical analyses for drug solubility and transfer rate assessment were performed by liquid chromatography equipped with a HILIC BEH amide column (50×3 mm, 1.7 μm, Waters, Manchester, UK) coupled to high resolution accurate mass spectrometry (Vanquish Duo-Q Exactive HF system, LC-HR-MS, ThermoFisher Scientific, Waltham, MA, USA). Mobile phases were composed of acetonitrile containing 0.1% formic acid and 10 mM ammonium formate. Samples were diluted to fit the calibration curve built from 9-calibrant levels (5-100 ng/mL). A volume of 5 μL diluted solution was injected. Mass spectrometry detection was realized in positive electrospray ionization with a mass resolution of 60,000 by scanning full scan mass from m/z 50-350.

Transfer rate assessments were performed to measure of the amount of chloroquine and hydroxychloroquine in aerosol delivered from the device containing the liquid formulation. A total of 30 puffs from 40 mg/mL chloroquine and 100 mg/mL hydroxychloroquine liquid formulation were collected on a Cambridge pad filter and the amount per puff was measured to be 149.69 and 330.32 μg respectively (FIG. 4B).

### Thermal Decomposition

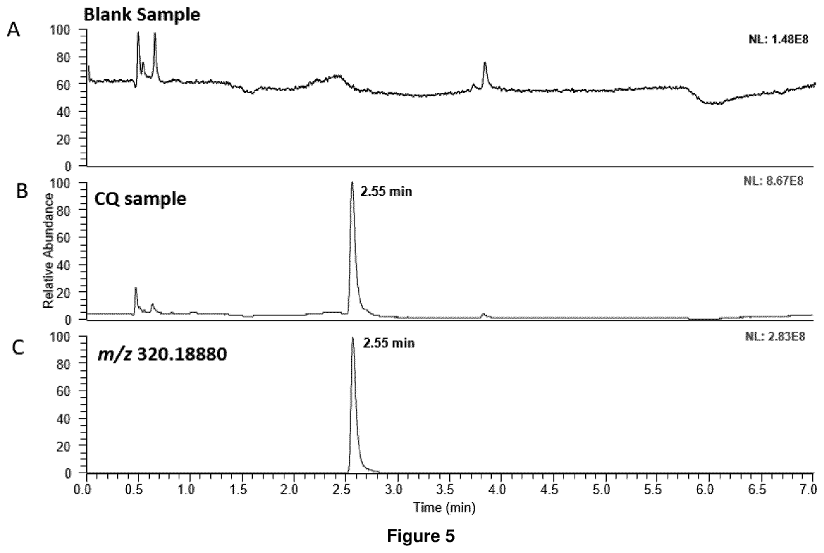

During thermal aerosolization there is a potential for decomposition however in the LC-HR-MS spectra only peaks representing chloroquine and hydroxychloroquine were observed, indicating no decomposition (FIG. 5).

### Cell Culture

Human 3D organotypic bronchial cultures were reconstituted from primary bronchial epithelial cells (Lonza, Basel, Switzerland) from a single donor. Cells were seeded on collagen !-coated Transwell® inserts (Corning®, Corning, NY, USA). Both apical and basal sides of the inserts were filled with PneumaCult™-EX PLUS medium (STEMCELL Technologies, Vancouver, Canada) and maintained for three days. Subsequently, the culture was air-lifted by removing the apical medium; the basal medium was replaced with the PneumaCult™-ALI medium (STEMCELL Technologies). Fully differentiated cultures were acclimatized in the incubator before exposure.

### Vitrocell Aerosol Exposure

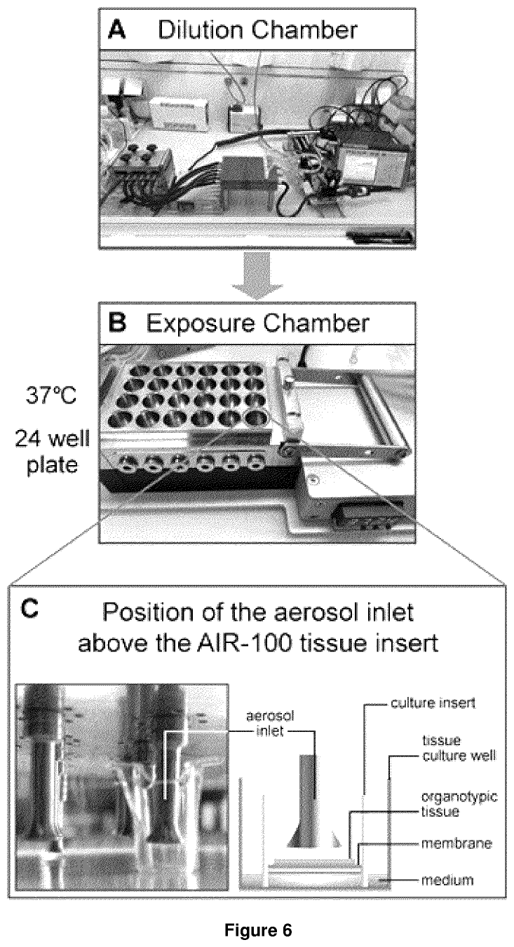

The Vitrocell® 24 exposure system (Vitrocell Systems GmbH, Waldkirch, Germany) and the PDSP pump (programmable dual syringe pump) were installed inside the chemical hood for the exposure of cell culture inserts (FIG. 6). A formulation containing a solution of chloroquine in propane-1,2-diol at a concentration of 25 mg/mL was prepared. Freshly generated aerosol was diluted and transferred via PDSP pump with a 55 mL puff volume, 3 second puff duration and a second puff interval to the exposure top and distributed into the Cultivation Base Module via port ejectors (trumpets) under negative pressure. A set of 3D organotypic human bronchial cultures were placed in the Cultivation Base Module and exposed to chloroquine aerosol on their apical side. The cell cultures were exposed to 25, 50 and 100 puffs of chloroquine or hydroxychloroquine aerosol, 100 puffs of air and 100 puffs of propylene glycol as a control. The compounds deposited in the exposure chamber were trapped using inserts containing ultra-pure water. The insert containing 110 microliters of ultra-pure water was placed in the Base Module of the Vitrocell® 24 exposure system and exposed together with the 3D organotypic cell cultures, in every exposure experiment. The concentrations of the deposited chloroquine and hydroxychloroquine were measured using liquid chromatography tandem-mass spectrometry. The amount of aerosolized chloroquine deposited in cell free controls was 7.24, 13.15 and 12.91 μg for 25, 50 and 100 puffs respectively (Table 2).

TABLE 2
Aerosol deposition in Vitrocell inserts. The liquid formulation
contained 2.5% of drug solubilized in propylene glycol (97.5%).

Chloroquine

| Number | Mean deposition | Percent |
|---|---|---|
| of puffs | (μg) ± SD | deposition (%) |
| 25 | 7.238 ± 0.840 | 0.23 |
| 50 | 13.148 ± 1.348 | 0.21 |

SD, standard deviation.

### 3D Human Bronchial Airway Culture

The potential adverse effect of chloroquine aerosol was assessed by exposing 3D human bronchial airway cultures to 25, 50 and 100 puffs of the aerosol generated from a formulation containing a solution of chloroquine in propane-1,2-diol at a concentration of 25 mg/mL. The functionality of 3D bronchial cultures was evaluated pre- and 24 hours post-exposure by measuring cilia beating frequency (CBF); cilia beating active area; transepithelial electrical resistance (TEER).

### Cell Viability

The viability of the 3D organotypic cultures 24 h post-exposure was evaluated by measuring the ATP content using the CellTiter-Glo® 3D Cell Viability Assay (Promega, Madison, WI, USA). CellTiter-Glo® reagent (150 μL) was added to the apical surface. After 30 minutes, 50 μL of CellTiter-Glo® reagent was transferred into an opaque-walled 96-well plate from the apical surface of the tissues, and luminescence in relative light units (RLU) was measured using a FLUOstar Omega plate reader (BMG Labtech, Ortenberg, Germany).

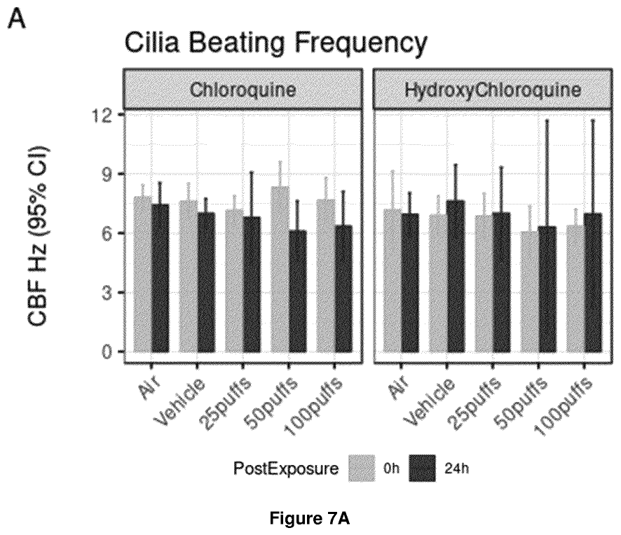

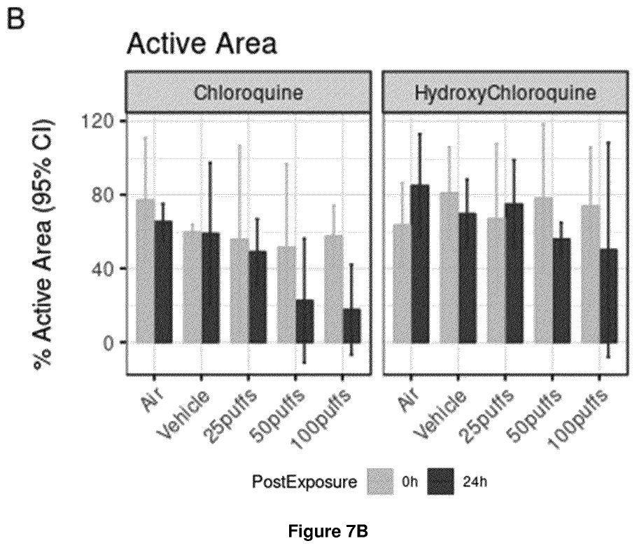

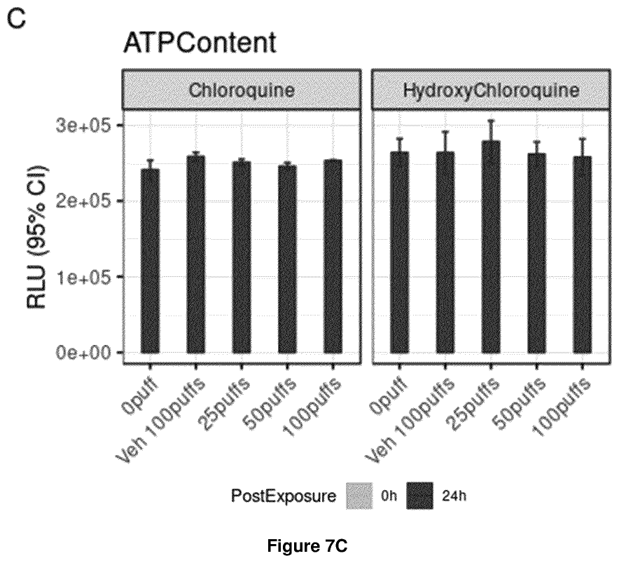

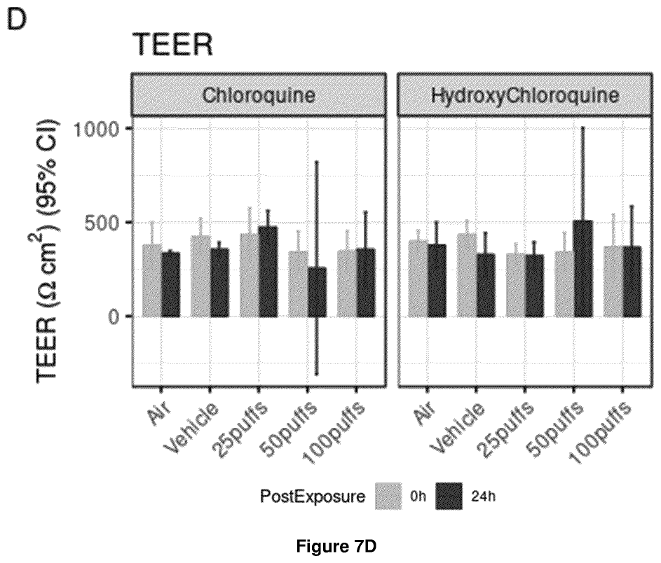

The viability was then assessed by measuring the ATP content present in the tissues 24 hours after exposure to chloroquine and hydroxychloroquine aerosol. For both the drugs and all the doses tested, the ATP content in tissues exposed to a drug (any dose) were similar to the ATP content measured in tissues exposed to the air or to the vehicle (FIG. 7C).

### Cilia Beating Frequency (CBF)

CBF and cilia beating active area measurements were conducted in 3D organotypic cultures using an inverted microscope (Zeiss, Oberkochen, Germany) equipped with a 4× objective and a 37° C. chamber and connected to a high-speed camera (Basler A G, Ahrensburg, Germany). Short movies composed of 512 frames recorded at 120 images per second were analyzed by using the SAVA analysis software (Ammons Engineering, Clio, MI, USA). The CBF of unexposed tissues ranged between 6 and 8 Hz for air and vehicle controls. The effect of chloroquine and hydroxychloroquine on CBF pre- and 24 hours post-exposure was compared with the results shown in FIG. 7A.

### Cilia Beating Active Area

Cilia beating active area corresponds to the percentage of the tissue surface where cilia beating were detected. The effect of chloroquine and hydroxychloroquine on cilia beating active area pre- and 24 hours post-exposure was compared with the results shown in FIG. 7B.

### Transepithelial Electrical Resistance (TEER)

TEER was measured in 3D organotypic cultures before the exposure and 24 h post-exposure using an EndOhm-6 chamber (WPI, Sarasota, FL, USA) connected to an EVOM™ Epithelial voltohmmeter (WPI), according to the manufacturer's instructions. The value displayed by the voltohmmeter was multiplied by the surface of the inserts (0.33 cm²) to obtain the resistance value in the total area (Ω×cm2). The TEER measurement performed to evaluate the human bronchial epithelium tightness showed that the electrical resistance ranged between 350 and 500 D*cm2 before and after exposure in all conditions tested (FIG. 7D). An exposure to 50 puffs of chloroquine had very low TEER values for two replicates post 24 h exposure while the third replicate had a TEER value similar to the value obtained before exposure. Moreover, an exposure to 100 puffs did not present a lowered TEER value and showed no overall effect.

### Modelling In Vitro and Isolated Perfused Mouse Lung Kinetics

The in vitro model consisted of apical mucus, periciliary layer, cytosol, lysosomal and basal compartments. The lysosomal compartment was nested in the cytosol compartment. Diffusion of the deposited compound across the mucus was calculated using the Hayduk-Laudie method (Gulliver J S. Introduction to Chemical Transport in the Environment. Cambridge: Cambridge University Press, 2007) by incorporating the viscosity of the airway mucus described in Lai S K, et al., Micro- and macrorheology of mucus. Adv Drug Deliv Rev 2009; 61(2): 86-100. The diffusive flux of diprotic bases between the periciliary layer and cytosol, cytosol and lysosome, and cytosol and basal compartments were implemented based on the model developed by Trapp et al. (Quantitative modelling of selective lysosomal targeting for drug design. Eur Biophys J 2008; 37(8): 1317-28). The drug transport across the compartments were the sum of the diffusive flux of neutral species calculated by the Fick's first law and ionic species by the Nernst Planck equation shown in eq. 1.

$$J = {f}_{n} {P}_{n} ( {C}_{n , o} - {C}_{n , i} ) + {f}_{dz} {P}_{dz} \frac{N}{{e}^{N} - 1} ( {C}_{dz , i} - {C}_{dz , i} {e}^{N} ) \qquad 1$$

where J, P, C, and N are the total diffusion flux, permeability, concentration and N= [symbol] EF/(RT); [symbol] is the electric charge (0 for neutral, +1 and +2 for ionic species), F is the faraday constant, E is the membrane potential, R is the real gas constant and T is the temperature. The subscripts n, d, o and I represent the fraction of species, neutral, ionic, outside and inside species. The neutral fraction of the drug f n available for diffusion was calculated from eq. 2, which accounts for water fraction (W), the lipid binding (L), sorption coefficients (K) and the ionic activity coefficients (y) for the compartment.

$${f}_{n} = \frac{1}{\frac{W + {K}_{n}}{{γ}_{n}} + \frac{{D}_{dz} W + {D}_{dz} {K}_{dz}}{{γ}_{dz}}} \qquad 2$$

A ratio of neutral and ionic fractions (D_{d} [symbol] ) of the compound in the given charged state (z) was calculated by using the Henderson-Hasselbalch equations, eqs. 3 and 4.

D_{d1}=10(pKₐ₁−pH) 3

D_{d2}=10(pKₐ₁+pKₐ₂−2pH) 4

In addition, the ionic activity coefficients (y) and the sorption coefficients (Kₙ and K_{d} [symbol] ) for neutral and ionic species were determined based on the lipophilicity, relative diffusivity factor (s) capturing the relative changes in organic compound specific diffusion coefficient, and cytosolic ionic strength (Iₒ). The equations to calculate permeability (P_{d} [symbol] ) of a given species are eqs. 5, 6, 7, 8 and 9.

$$\begin{matrix}{γ}_{n} = {10}^{0.3 * {I}_{o}} & 5 \\ {γ}_{dz} = {10}^{\frac{- 0.5 z \sqrt{{I}_{o}}}{1 + \sqrt{{I}_{o} - 0.3 * {I}_{o}}}} & 6 \\ log {P}_{dz} = {10}^{log ( logP ) - 3.5 z} & 7 \\ {K}_{dz} = 1.22 * L * log {P}_{dz} & 8 \\ {P}_{dz} = {10}^{log ( {logP}_{dz} - Δ s )} & 9\end{matrix}$$

Earlier studies from Ohkuma S, et al. (Fluorescence probe measurement of the intralysosomal pH in living cells and the perturbation of pH by various agents. Proc Natl Acad Sci USA 1978; 75(7): 3327-31) and Reijngoud D J et al. (Chloroquine accumulation in isolated rat liver lysosomes. FEBS Lett 1976; 64(1): 231-5) have indicated that accumulation of diprotic weak bases have an impact of lysosomal pH even in the presence of the lysosomal buffering. The changes in the lysosomal pH was included as a dynamic compartment using a linear eq. 10,

$${pH}_{lys} = {pH}_{lys , t = 0} - \frac{{C}_{lys}}{β} \qquad 10$$

where, pH_{lys,t=0} is the initial pH of the lysosome, C_{lys} is the concentration of drug in the lysosome and β is the lysosomal buffering capacity according to Collins K P et al. (Hydroxychloroquine: A Physiologically-Based Pharmacokinetic Model in the Context of Cancer-Related Autophagy Modulation. J Pharmacol Exp Ther 2018; 365(3): 447-59) and Ishizaki J. et al. (Uptake of imipramine in rat liver lysosomes in vitro and its inhibition by basic drugs. J Pharmacol Exp Ther 2000; 294(3): 1088-98). The model also incorporated an active transport of compounds from cytosol to periciliary layer via the P-gp efflux transporter and was modeled using the parameters obtained from Price et. al. (The Differential Absorption of a Series of P-Glycoprotein Substrates in Isolated Perfused Lungs from Mdr1a/1b Genetic Knockout Mice can be Attributed to Distinct Physico-Chemical Properties: an Insight into Predicting Transporter-Mediated, Pulmonary Specific Disposition. Pharm Res 2017; 34(12): 2498-516). The differential equations describing the changes in concentrations of compartments representing the human bronchial epithelium at ALI were eqs. 11, 12, 13, 14 and 15,

$$\begin{matrix}\frac{d}{dt} {C}_{muc} = \frac{1}{{V}_{muc}} * ( - \frac{D * {SA}_{tissue}}{{T}_{muc}} * ( {C}_{muc} - {C}_{pcl} ) ) & 11 \\ \frac{d}{dt} {C}_{pcl} = \frac{1}{{V}_{pcl}} * ( \frac{D * {SA}_{tissue}}{{T}_{pcl}} ( {C}_{muc} - {C}_{pcl} ) - {SA}_{tissue} ( {J}_{pcl - cyt} * {C}_{pcl} - {J}_{cyt - pcl} * {C}_{cyt} ) + \frac{{Vmax}_{pgp} * {C}_{cyt}}{{Km}_{pgp} + {C}_{cyt}} ) & 12 \\ \frac{d}{dt} {C}_{cyt} = \frac{1}{{V}_{cyt} - {V}_{lys}} * ( {SA}_{tissue} ( {J}_{pcl - cyt} * {C}_{pcl} - {J}_{cyt - pcl} * {C}_{cyt} ) + {SA}_{tissue} ( {J}_{bas - cyt} * {C}_{bas} - {J}_{cyt - bas} * {C}_{cyt} ) - {SA}_{lys} ( {J}_{cyt - lys} * {C}_{cyt} - {J}_{lys - cyt} * {C}_{lys} ) - \frac{{Vmax}_{pgp} * {C}_{cyt}}{{Km}_{pgp} + {C}_{cyt}} ) & 13 \\ \frac{d}{dt} {C}_{lys} = \frac{1}{{V}_{lys}} * ( {SA}_{lys} ( {J}_{cyt - lys} * {C}_{cyt} - {J}_{lys - cyt} * {C}_{lys} ) ) & 14 \\ \frac{d}{dt} {C}_{bas} = \frac{1}{{V}_{bas}} * ( - {SA}_{tissue} ( {J}_{bas - cyt} * {C}_{bas} - {J}_{cyt - bas} * {C}_{cyt} ) ) & 15\end{matrix}$$

where C, D, SA, T and V are concentration, diffusion coefficient, surface rea, thickness and volume of the compartment. The subscripts muc, pcl, tissue, lys and bas represent the mucus, periciliary layer, cytosol, lysosome and basolateral compartments.

Similarly, an in silico model representing the pulmonary alveolar region of an isolated perfused mouse lung (IPML) was developed with 6 compartments. The transport kinetics had a similar formalism to the in vitro model and described the concentration changes in compartments, namely surfactant, cytosol, lysosome, interstitial, vascular and perfusate. During the flow of perfusate, an instantaneous equilibrium was assumed between the vascular and interstitial compartments for unbound drug concentrations.

The transport kinetics of chloroquine and hydroxychloroquine across the airway epithelium were modelled for hydroxychloroquine in 3D organotypic human bronchial epithelial cultures (HBEC) at air-liquid interface and for chloroquine in an isolated perfused mouse lung. Since, the in vitro airway surface liquid in human bronchial epithelial cells was measured to be acidic, a pH of 6.8 was set to the apical mucus and periciliary layer compartments (Saint-Criq V, et al., Real-Time, Semi-Automated Fluorescent Measurement of the Airway Surface Liquid pH of Primary Human Airway Epithelial Cells. J Vis Exp. 2019(148).

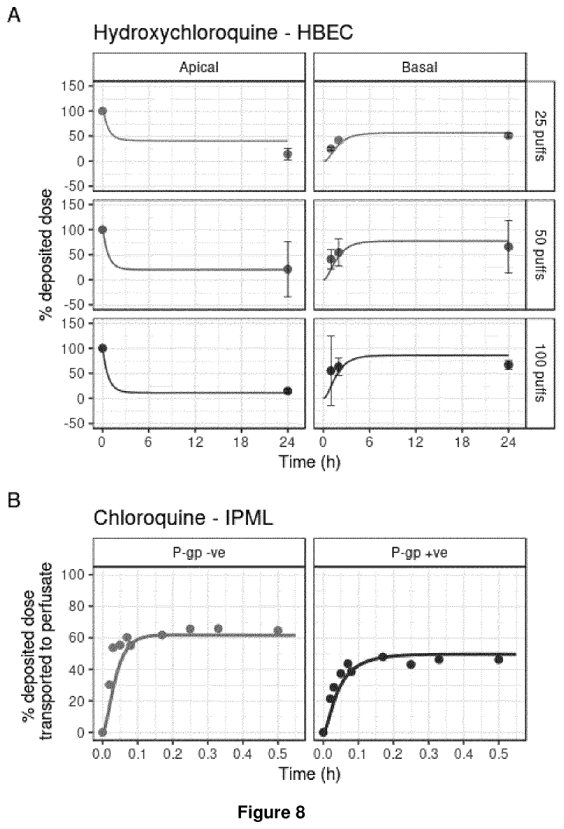

Although the apically deposited hydroxychloroquine reached equilibrium across different compartments, after 6 h post exposure a 54.72%, 75.65% and 84.28% of the deposited dose was transferred to the basal compartment while 40.65%, 19.75% and 11.19% of the deposited dose remained in the apical compartment for 25, 50 and 100 puffs respectively (FIG. 8A). The difference in fractions of compound in apical and basal compartments for different puffs of exposure is due to pH dependent lysosomal trapping of hydroxychloroquine.

The experimental data and parameters used to model the transport kinetics of aerosolized CQ in IPML were obtained from Price et. al (The Differential Absorption of a Series of P-Glycoprotein Substrates in Isolated Perfused Lungs from Mdr1a/1b Genetic Knockout Mice can be Attributed to Distinct Physico-Chemical Properties: an Insight into Predicting Transporter-Mediated, Pulmonary Specific Disposition. Pharm Res. 2017; 34(12):2498-2516). The reported aerosol deposited fraction in the IMPL was 80% of the delivered dose. In mice, the physiologically relevant pH of airway surface liquid and cytosol were set at 7.1 and 6.8 respectively (Brown R P, et al., Physiological parameter values for physiologically based pharmacokinetic models, Toxicol Ind Health 1997; 13(4): 407-84 and Sarangapani R, et al., Physiologically based pharmacokinetic modeling of styrene and styrene oxide respiratory-tract dosimetry in rodents and humans, Inhal Toxicol 2002; 14(8): 789-834). The model was simulated with and without the P-gp efflux transporter, using the reported values for chloroquine (Price et al.). In P-gp knockout IPML, 61.68% of the deposited dose was transported across the pulmonary barrier in 13.36 min (FIG. 8B). Whereas in an active P-gp efflux IPML, the percentage of compound in the perfusate media was 11.97% lower than that of the P-gp knockout IPML. Because the IMPL experimental system was a closed loop system where the perfusate was recirculated, an equilibrium between the pulmonary airway epithelium and perfusate was attained.

### PBPK Modelling

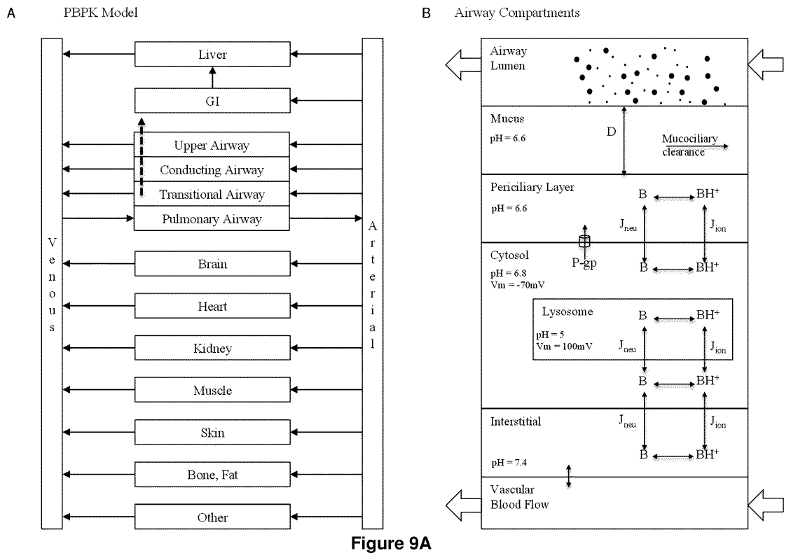

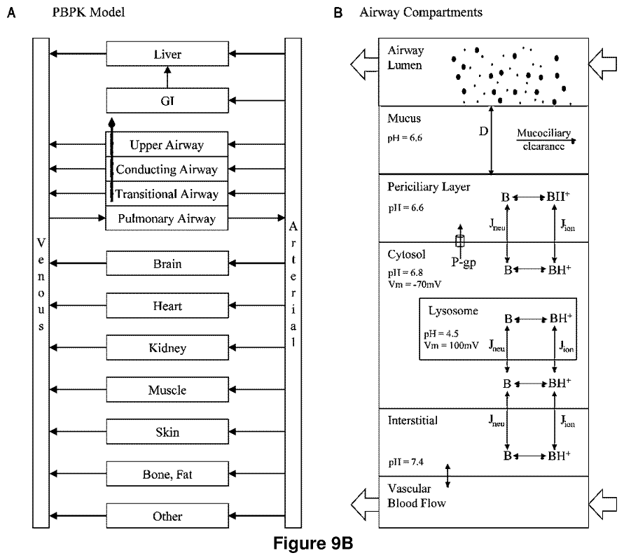

A flow-limited PBPK model of chloroquine consisting of 16 tissue compartments, including the regional respiratory tract compartments was developed (FIG. 9). In addition, the lysosomal compartment for each tissue was nested and the kinetics of lysosomal trapping was implemented based on the in vitro model in Trapp et al. Also, as described for the in vitro model, the nested lysosomal compartmental pH was dynamic. A general mass balance equation along with the lysosomal kinetics for a single tissue compartment are described by eqs. 16 and 17

$$\begin{matrix}\frac{dx}{dt} {C}_{tissue} = \frac{1}{{V}_{tissue}} ( {Q}_{tissue} ( {C}_{art} - \frac{{C}_{tissue}}{{PN}_{tissue}} ) - {SA}_{tissuelys} ( {J}_{tissuelys} * {C}_{tissue} - {J}_{lystissue} * {C}_{tissuelys} ) ) & 16 \\ \frac{dx}{dt} {C}_{tissuelys} = \frac{1}{{V}_{tissuelys}} ( {SA}_{tissuelys} ( {J}_{tissuelys} * {C}_{tissue} - {J}_{lystissue} * {C}_{tissuelys} ) ) & 17\end{matrix}$$

where Cₐᵣₜ, Cₜᵢₛₛᵤₑ, C_{tissuelys}, Q, V, SA_{tissuelys} and J are the arterial, tissue, tissue specific lysosomal concentrations, blood flow rate, volume of the compartment, surface area of the lysosome and diffusive flux respectively. The respiratory tract (RT) was divided into 4 regions based on the anatomical location and function described in Sarangapani R, et al. The model consisted of the upper airways (nose, mouth and larynx), conducting airways (airway branching from generation 0-10), transitional airways (airway branching from generation 11-16) and pulmonary airways (airway branching from generations 17-24). Each respiratory tract regions were modelled in detail by further dividing them into 6 compartments representing the mucus, periciliary layer, cytosol, lysosomal, interstitial space and vascular space. Since the pulmonary airways do not contain mucus and periciliary layer, a single compartment representing the surfactant layer was included. In addition, mucociliary clearance from transitional, conductional and upper airways to gastrointestinal tract was modeled using the rates obtained from Ashgarian et al. (Mucociliary clearance of insoluble particles from the tracheobronchial airways of the human lung. Journal of Aerosol Science 2001; 32(6): 817-32). Using the above framework, PBPK models for mouse, rat and human were developed. The physicochemical parameters for chloroquine were obtained from literature and were used to predict the partitioning coefficients of diprotic bases by Rodger's method (Rodgers T, Leahy D, Rowland M. Physiologically based pharmacokinetic modeling 1: predicting the tissue distribution of moderate-to-strong bases. Journal of pharmaceutical sciences 2005; 94(6): 1259-76). While the physiological tissue volumes and blood flow rates were standard values from Brown et al. the respiratory tract descriptions were obtained from Sarangapani et al. The PBPK model was constructed and simulated in R language (Version 3.5.1) using R packages such as ‘mrgsolve’ (Baron K T, et al., Simulation from ODE-based population PK/PD and systems pharmacology models in R with mrgsolve. Omega 2015; 2: 1×) for describing the PBPK framework, GenSA (Xiang Y, et al., Generalized Simulated Annealing for Global Optimization: The GenSA Package. R Journal 2013; 5(1)) for model optimization and rggplot2′ (Wickham H. ggplot2: elegant graphics for data analysis: springer, 2016) for generating plots. The plasma and tissue time concentrations from different publications were obtained by digitizing graphs using WebPlotDigitizer (Rohatgi A. WebPlotDigitizer. Austin, Texas, USA, 2017). Model optimization was performed by minimizing the residual sum of squares.

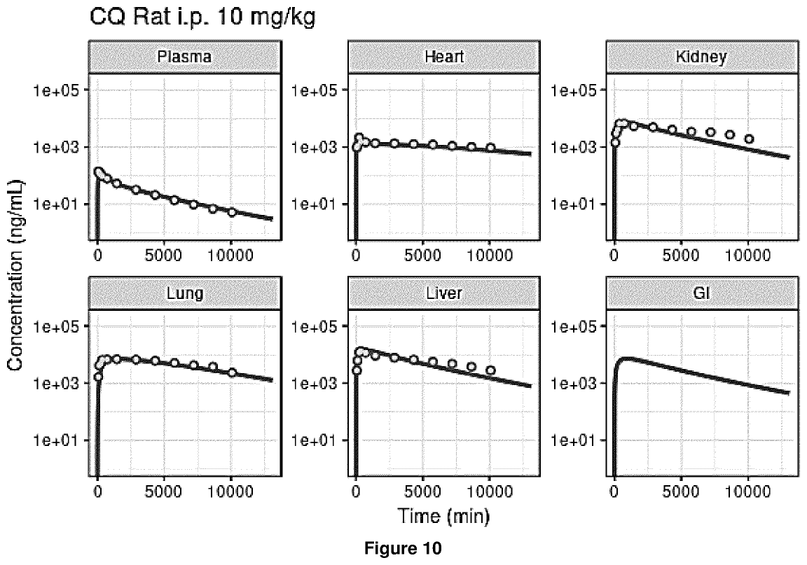

The schematic for the PBPK model developed is shown in FIG. 9A. To predict physiologically relevant lung concentrations a mechanistic model describing the transport kinetics across the airway epithelium was included FIG. 9B. The predicted and observed plasma and tissue concentrations of chloroquine in rats is shown in FIG. 10. Upon intraperitoneal (i.p) administration of 10 mg/kg chloroquine to rats, the plasma Cₘₐₓ and terminal elimination half-life were 0.15 pg/mL and 14.6 h respectively while the lung tissue exposures were significantly higher with a Cₘₐₓ of 8.39 pg/mL and a half-life of 125.2 h because of lysosomal trapping. The difference in lung tissue elimination half-life for both the compounds is significantly different not only due to physicochemical properties but also due to the physiological differences in lung across species.

For the human PBPK model, the airway surface fluid and intracellular epithelial pH were set to be acidic with pHs of 6.6 (Bodem C R et al., Endobronchial pH. Relevance of aminoglycoside activity in gram-negative bacillary pneumonia. Am Rev Respir Dis. 1983; 127(1):39-41) and 6.8 (Paradiso A M et al., Polarized distribution of HCO3-transport in human normal and cystic fibrosis nasal epithelia. J Physiol. 2003; 548(Pt 1):203-218). Since the intralysosomal pH in human lung is not known, a pH value of 4.5 obtained from baboon was used (Hellmann P et al., Intraphagolysosomal pH in canine and rat alveolar macrophages: flow cytometric measurements. Environ Health Perspect. 1992; 97:115-120).

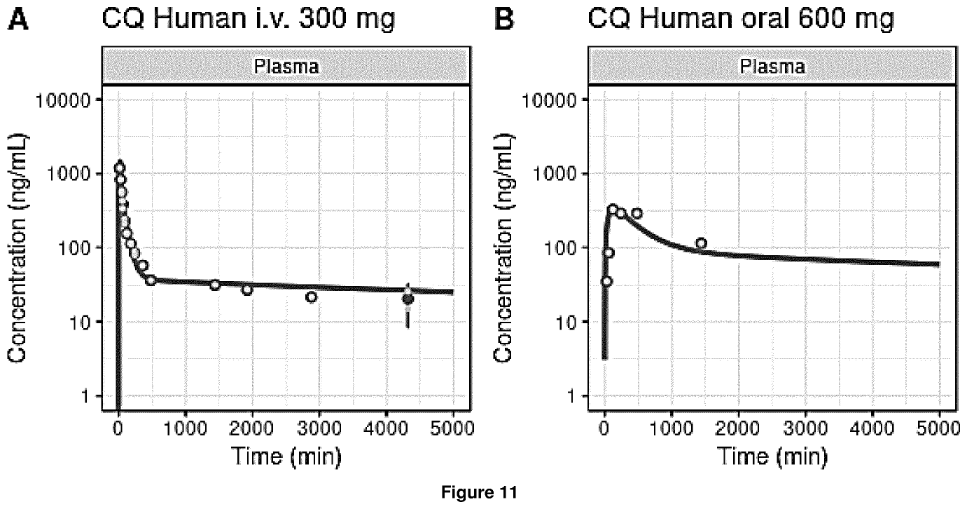

The pharmacokinetics of chloroquine were validated to intravenous and oral dosing data obtained from Gustafsson et al. The predicted plasma concentration time profiles for chloroquine administered intravenously and orally are shown in FIG. 11. The plasma terminal elimination half-lives for chloroquine was 158.12 h.

The validated human PBPK model was employed to simulate the concentration time profiles of oral dosing regimens of chloroquine used for treating COVID-19.

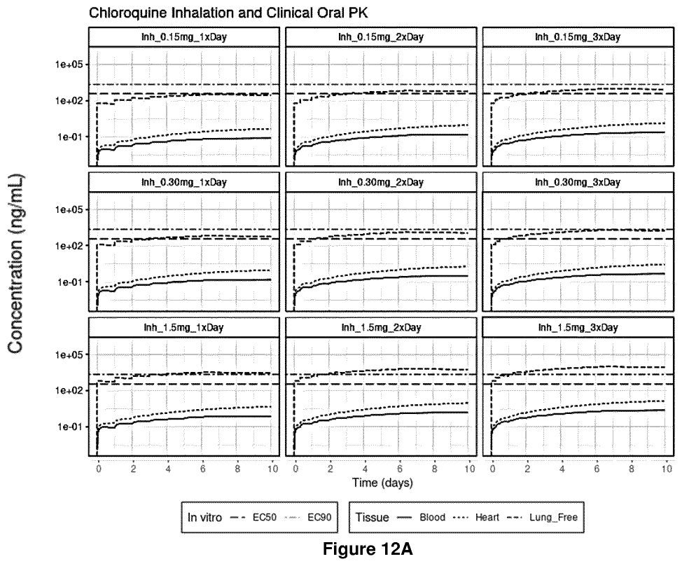

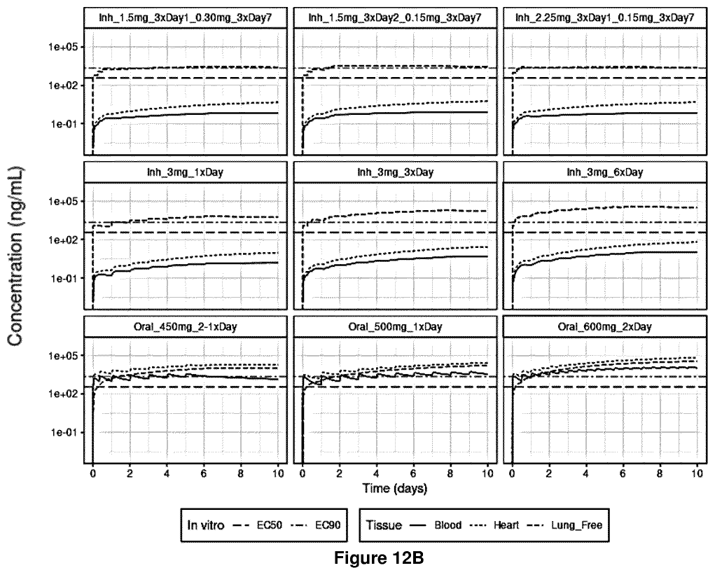

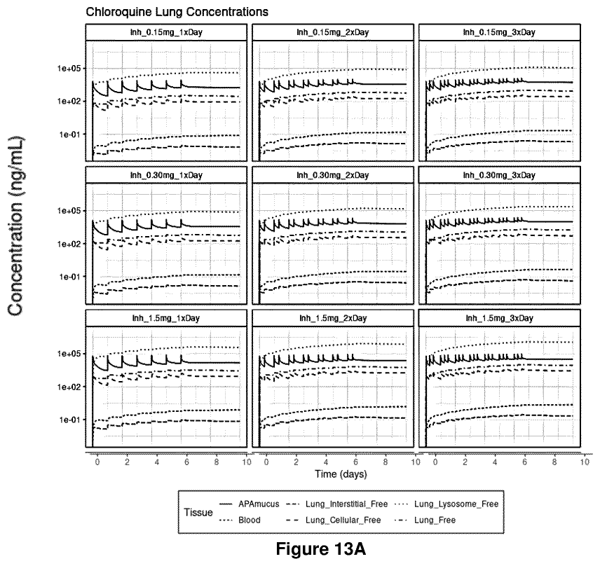

Clinically administered oral dosing profiles for chloroquine with a dosing regimen of 450 mg b.i.d on day 1 and 450 mg q.d from day 2 to day 5, is shown in FIG. 12 (bottom row, 1^{st} column). Although, the total lung unbound concentrations for oral dosing achieved the in vitro EC₅₀ values reported by Wang et al. and Yao et al. the dosing regimen also increased the accumulation of chloroquine in tissues such as heart, liver, kidney, etc., thus limiting the ability to deliver higher doses or prolonged usage to further increase lung concentrations.

The validated human PBPK model was also employed to simulate the concentration time profiles of orally inhaled dosing regimens of chloroquine used for treating COVID-19.

For an orally inhaled aerosol, the multiple-path particle dosimetry model predicted a 28.97% deposition and a 71.03% exhaled fraction per puff based on the measured aerosol physicochemical properties. The regional deposition fractions per puff were 1.19, 3.05, 5.08 and 19.64% in the upper airways, conducting airways, transitional airways and pulmonary airways respectively. A puffing pattern of a 3 second inhalation-exhalation with a 30 second inter-puff interval was used in the simulation based on a 40 mg/mL chloroquine liquid formulation 55 mL puff volume. Multiple inhalation dosing regimens with an inhaled dose of 0.15 mg/puff chloroquine with multiple puffs/session/day were simulated to predict the inhalation PK (FIG. 9). The basis for inhalation dosing regimen selection was to attain unbound lung trough concentrations equal to or greater than the in vitro EC₅₀ and EC₉₀ values defined in Wang et al. with respect to effective lung concentrations from an oral dose. The dosing simulations were based on a 70 kg subject.

A daily low inhalation dose consisting of one to three puffs of 0.15 mg/puff of chloroquine enables us to achieve the unbound lung concentrations to reach in vitro EC₅₀ values within a few days from start of treatment (FIG. 12).

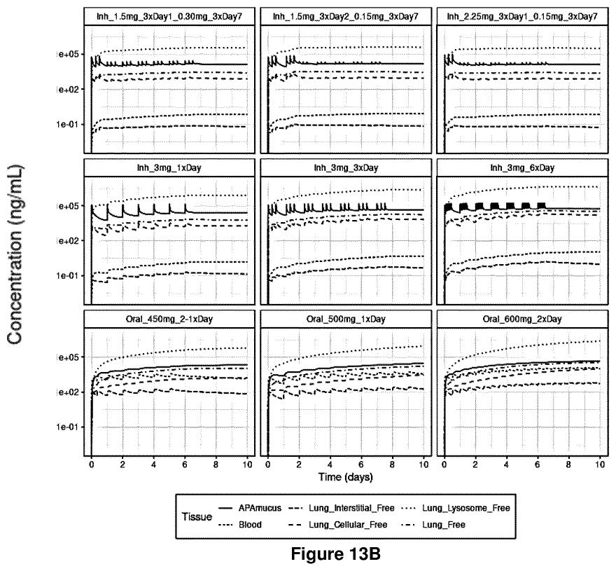

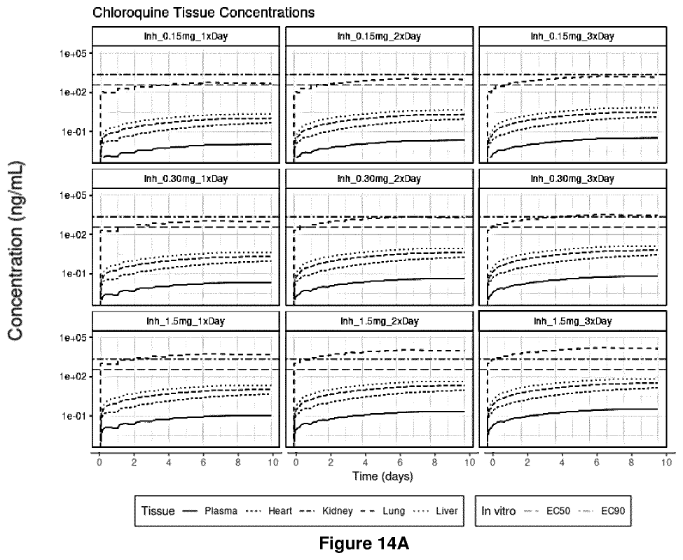

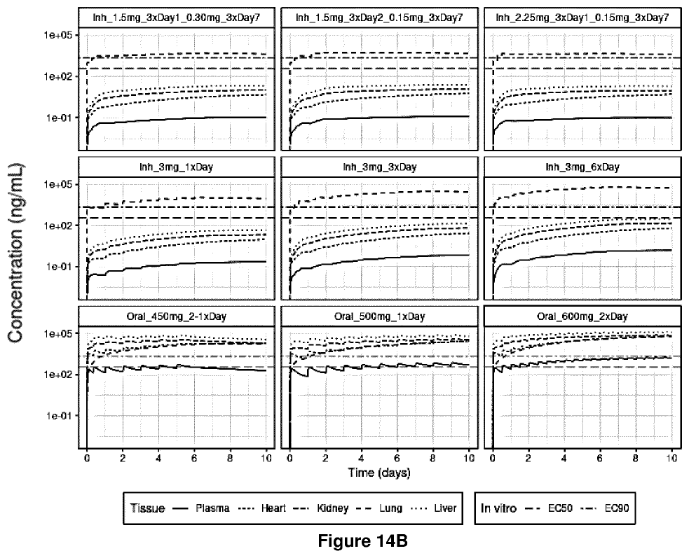

Alternatively, the unbound lung concentrations could reach in vitro EC₉₀ concentrations with a loading dose of 10 puffs (0.15 mg/puff) taken 3 times on day 1 followed by a maintenance dose of 1 puff taken 3 times a day on day 2 to day 7. Simulation of other dosing regimens include delivery of higher doses can be found in FIG. 12. Because the pharmacokinetic driver for the efficacy of chloroquine in the lung tissue is not clear, the concentration vs time profiles of drug in different compartments of the lung namely, mucus, periciliary layer, cytosol, lysosomes, interstitial fluid and vascular space can be found in FIG. 13.

Oral dosing regimens were designed for the plasma concentration to stay below 400 ng/mL as no adverse reactions are observed at this level. A warning limit of 800 ng/mL was also set at which 80% of the subjects showed adverse effects (Frisk-Holmberg M, et al., Chloroquine serum concentration and side effects: evidence for dose-dependent kinetics; Clin Pharmacol Ther 1979; 25:345-50 and Cui et al., Dose selection of chloroquine phosphate for treatment of COVID-19 based on a physiologically based pharmacokinetic model; Acta Pharma. Sinica B, Vol. 10, Iss. 7, July 2020). However, although the plasma concentrations for the oral dosing regimens remained close to the 400 ng/mL limit, the concentrations in liver, heart and kidney were significantly higher. In contrast, all of the orally inhaled dosing regimens showed plasma, liver, heart and kidney concentrations well below the 800 ng/mL and 400 ng/mL limits.

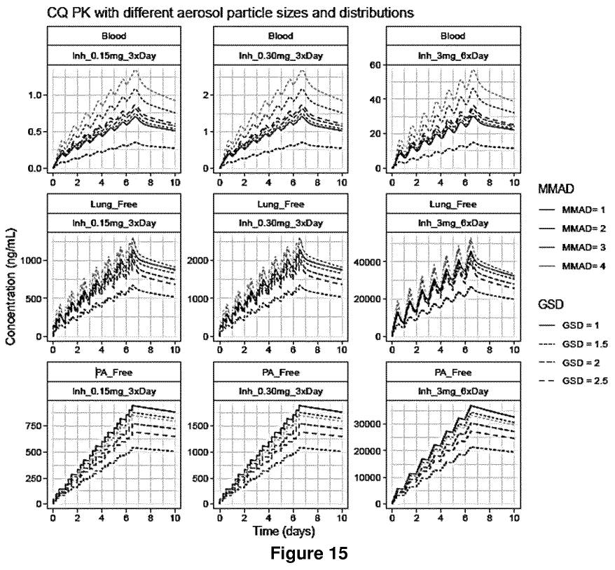

Since aerosol particle sizes influence the regional deposition of an inhaled aerosol Anjilvel S, et al. (A multiple-path model of particle deposition in the rat lung. Fundam Appl Toxicol. 1995; 28(1):41-50.) and Kolli A R, et al. (Bridging inhaled aerosol dosimetry to physiologically based pharmacokinetic modeling for toxicological assessment: nicotine delivery systems and beyond. Crit Rev Toxicol. 2019; 49(9):725-741), a simulation of inhalation PK for monodisperse and polydisperse aerosols was performed (FIG. 15). An increase in mass median aerodynamic diameter (MMAD) had led to a rise in systemic concentrations while the pulmonary alveolar concentrations were influenced by a combination of MMAD (1-3 μm) and geometric standard deviation (1-1.5).

TABLE 3
Description of simulated CQ dosing regimens
administered via oral and inhalation routes.

| | Dose per | | |
|---|---|---|---|
| Dosing regimen | day | | Description |
| Inh_0.15 mg_1xDay | 0.15 | mg | Inhalation of 0.15 mg/puff, 1 |
| | | | puff/session, 1 session/day, |
| | | | 24 hr gap/session |
| Inh_0.15 mg_2xDay | 0.30 | mg | Inhalation of 0.15 mg/puff, 1 |
| | | | puffs/session, 2 session/day, |
| | | | 8 hr gap/session (b.i.d dosing) |
| Inh_0.15 mg_3xDay | 0.75 | mg | Inhalation of 0.15 mg/puff, 1 |
| | | | puffs/session, 3 session/day, |
| | | | 6 hr gap/session (t.i.d dosing) |
| Inh_0.30 mg_1xDay | 0.30 | mg | Inhalation of 0.15 mg/puff, 2 |
| | | | puff/session, 1 session/day, |
| | | | 24 hr gap/session |
| Inh_0.30_2xDay | 0.60 | mg | Inhalation of 0.15 mg/puff, 2 |
| | | | puffs/session, 2 session/day, |
| | | | 8 hr gap/session (b.i.d dosing) |
| Inh_0.30_3xDay | 0.90 | mg | Inhalation of 0.15 mg/puff, 2 |
| | | | puffs/session, 3 session/day, |
| | | | 6 hr gap/session (t.i.d dosing) |
| Inh_1.5 mg_ 1xDay | 1.5 | mg | Inhalation of 0.15 mg/puff, 10 |
| | | | puff/session, 1 session/day, |
| | | | 24 hr gap/session |
| Inh_1.5 mg_2xDay | 3.0 | mg | Inhalation of 0.15 mg/puff, 10 |
| | | | puffs/session, 2 session/day, |
| | | | 8 hr gap/session (b.i.d dosing) |
| Inh_1.5 mg_3xDay | 4.5 | mg | Inhalation of 0.15 mg/puff, 10 |
| | | | puffs/session, 3 session/day, |
| | | | 6 hr gap/session (t.i.d dosing) |
| Inh_1.5 mg_3xDay | 1.4 mg (9.9 | | Inhalation of 0.15 mg/puff, 10 |
| 1_0.30 mg_3xDay 7 | mg for 7 days) | | puffs/session, 3 sessions/day, |
| | | | 6 hr gap/session on day 1 and 2 |
| | | | puffs/session, 3 sessions/day, |
| | | | 6 hr gap/session from day 2 to |
| | | | day 7 |
| Inh_1.5 mg_3xDay | 1.6 mg (11.3 | | Inhalation of 0.15 mg/puff, 10 |
| 2_0.15 mg_3xDay 7 | mg for 7 days) | | puffs/session, 3 sessions/day, |
| | | | 6 hr gap/session on day 1 and |
| | | | day 2 followed by 1 |
| | | | puffs/session, 3 sessions/day, |
| | | | 6 hr gap/session from day 3 to |
| | | | day 7 |
| Inh_2.25 mg_3xDay | 1.35 mg (9.45 | | Inhalation of 0.15 mg/puff, 15 |
| 1_0.15 mg_3xDay 7 | mg for 7 days) | | puffs/session, 3 sessions/day, |
| | | | 6 hr gap/session on day 1 |
| | | | followed by 1 puffs/session, 3 |
| | | | sessions/day, 6 hr gap/session |
| | | | from day 3 to day 7 |
| Inh_3 mg_1xDay | 3.0 | mg | Inhalation of 0.15 mg/puff, 20 |
| | | | puff/session, 1 session/day, |
| | | | 24 hr gap/session |
| Inh_3 mg_3xDay | 9.0 | mg | Inhalation of 0.15 mg/puff, 20 |
| | | | puffs/session, 3 session/day, |
| | | | 6 hr gap/session (t.i.d dosing) |
| Inh_3 mg_6xDay | 18.0 | mg | Inhalation of 0.15 mg/puff, 20 |
| | | | puffs/session, 6 session/day, |
| | | | 2 hr gap/session |
| Oral_500 mg_1xDay | 500 | mg | Oral dosing of 500 mg once a |
| | | | day (q.d) |
| Oral_600 mg_2xDay | 1200 | mg | Oral dosing of 600 mg twice a |
| | | | day (b.i.d) |

| | | |
|---|---|---|
| Oral_450 mg_2- | 385 mg (2700 | Oral dosing of 450 mg b.i.d on |
| 1xDay | mg total dose | day 1 and 450 mg q.d from day |
| | for 5 days) | 2 to day 5 |

1.-24. (canceled)

25. A pharmaceutical composition, comprising:
chloroquine or a pharmaceutically acceptable salt thereof; and
a solvent,
wherein the chloroquine or salt is present in a range of from 1 to 200 mg/mL,
wherein the solvent is propylene glycol, glycerine, propane-1,3-diol, water, or a combination thereof, and
wherein the pharmaceutical composition is suitable for thermal aerosolization.

26. The composition of claim 25, wherein the chloroquine or salt is present in a range of from 1 to 50 mg/mL.

27. The composition of claim 25, wherein the chloroquine or salt is present in a range of from 10 to 45 mg/mL.

28. The composition of claim 25, which is suitable for thermal aerosolization.

29. A pharmaceutical composition, comprising:
chloroquine or a pharmaceutically acceptable salt thereof suitable for use in the treatment or prevention of a viral lung infection,
wherein the pharmaceutical composition is suitable to be administered by oral inhalation.

30. The composition of claim 29, wherein the viral lung infection is caused by Betacoronavirus.

31. The composition of claim 29, which is suitable to be administered as a daily dose.

32. The composition of claim 31, wherein the daily dose comprises 0.001 mg to 20 mg of the chloroquine or salt.

33. The composition of claim 31, wherein the daily dose is selected from a loading dose, a maintenance dose, or combination thereof.

34. The composition of claim 33, wherein the loading dose is present and comprises 1 mg to 20 mg of the chloroquine or salt.

35. The composition of claim 33, wherein the maintenance dose is present and comprises 0.001 mg to 10 mg of the chloroquine or salt.

36. The composition of claim 33, wherein at least one of the loading dose is followed by at least one of the maintenance dose.

37. The composition of claim 29, wherein the daily dose is administered in at least one session.

38. The composition of claim 29, wherein the daily dose is administered in at least two sessions separated by intervals of up to twelve hours.

39. The composition of claim 37, wherein the at least one session comprises at least one fixed dose.

40. The composition of claim 39, wherein the fixed dose is a metered dose.

41. The composition of claim 29, comprised in a liquid aerosol.

42. The composition of claim 41, wherein the liquid aerosol has a Mass Median Aerodynamic Diameter (MMAD) in a range of from 1 to 5 μm.

43. The composition of claim 29, configured for administration to a mammalian or avian subject.

44. The composition of claim 29, configured for administration to a human subject and/or the subject is at risk of having COVID-19.
